(19) United States
(12) Patent Application Publication
(10) Pub. No.: US 2015/0160229 A1
(43) Pub. Date: Jun. 11, 2015

(54) METHODS FOR DETECTION OF HEART FAILURE

(71) Applicants: Thomas D. SCHAAL, San Francisco, CA (US); Robert J. SCHOTT, San Diego, CA (US); Yan LIU, Foster City, CA (US); Erhard Ralf SCHOENBRUNNER, Moraga, CA (US); Thomas A. LITTLE, Highland, UT (US)

(72) Inventors: Thomas D. SCHAAL, San Francisco, CA (US); Robert J. SCHOTT, San Diego, CA (US); Yan LIU, Foster City, CA (US); Erhard Ralf SCHOENBRUNNER, Moraga, CA (US); Thomas A. LITTLE, Highland, UT (US)

(21) Appl. No.: 14/563,929

(22) Filed: Dec. 8, 2014

Related U.S. Application Data
(60) Provisional application No. 61/913,212, filed on Dec. 6, 2013.

(51) Int. Cl. G01N 33/68

(52) U.S. Cl. CPC G01N 33/68; G01N 2333/92

(57) ABSTRACT

Described herein are methods for diagnosing and/or treating an individual for heart failure based on the individual's level of Lp-PLA2, or the individual's level of Lp-PLA2 and GDF-15, or the individual's level of Lp-PLA2 and sST2.

### CROSS REFERENCE TO RELATED APPLICATION

This application claims priority to U.S. Provisional Patent Application No. 61/913,212, filed on Dec. 6, 2013, and titled “BIOMARKERS FOR HEART FAILURE,” which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are compositions, kits, and methods using one or, more preferably, two or more, biomarkers for treating (including diagnosing, identifying, and prognosticating) heart failure.

### BACKGROUND

The incidences of heart failure (HF) are increasing in the developed world. Heart failure is not the result of one single disease state, but, rather it is a complex syndrome spanning a broad range of pathophysiological features including myocyte injury/stress, inflammation/oxidative stress, neurohormonal responses to decompensation, extracellular matrix remodeling, and renal dysfunction. Heart failure is currently determined using a variety of tests to place the degree of heart failure into one of four classes from I to IV using the New York Heart Association (NYHA) Functional Classification system. In this system, Class I heart failure is the least severe, with no symptoms of heart failure and class IV is the most severe. Tests used for classifying heart failure may include analysis of a blood sample that is tested increased levels of B-type natriuretic peptide (BNP) which is indicative of heart failure. Placing heart failure into one of these classes aids in determining what treatment should be given. Early and appropriate intervention leads to the best outcomes. Although heart failure is common, its diagnosis is often missed. It may be missed, for example, because a person may have no symptoms (e.g. such as found in NYHA Class I heart failure), or may be diagnosed with another disease with similar symptoms, or because a test is dangerous, expensive, unavailable, or gives ambiguous or false results.

Existing diagnostic tests may also be problematic. For example, BNP/pro-BNP tests may be less reliable in obese patients or patients with renal failure. Thus, there is a need for more reliable assays and treatment methods, as well as more effective markers to identify and stratify individuals at risk for heart failure or having heart failure.

LpPLA2 has been previously proposed as a that biomarker for use only in predicting outcomes for patient's already diagnosed with heart failure and only for patient's within NYHA class III and IV. See, e.g., Gerber, Y., et al. Plasma lipoprotein-associated phospholipase A2 levels in heart failure: Association with mortality in the community. Atherosclerosis 203 (2009) 593-598; Van Vark, L. C., et al. Lipoprotein-associated phospholipase A2 activity and risk of heart failure: the Rotterdam Study. European Heart Journal (2006) 27, 2346-2352; and Schott and Berg, Biomarkers in Heart Failure: Lp-PLA2 (activity) was predictive of incident heart failure in an at-risk population and was prognostic in a population with heart failure (Lp-PLA2 mass). Further, these studies examined only patient's already identified as late-stage heart failure patients, and failed to identify any significant effect in otherwise healthy patients (e.g., patients within NYHA class I and II). For example, the Gerber et al. paper specifically references only NYHA class >3, and even then shows only a dubious statistical significance (p=0.26; See Table 1 of Gerber. Importantly, Gerber and colleagues do not show any evidence for utilizing Lp-PLA2 (mass) analyte levels to distinguish Heart Failure patients from patients not suffering from Heart Failure, or to discriminate between patients with different NYHA classes of disease severity.

Described herein are techniques that may be particularly useful for diagnosing even the early stages of CHF prognosis (e.g., NYHA classes I and II). Specifically, the techniques described herein may be used to predict/prognose increased risk for apparently healthy individuals to progress to Stage I or II heart failure.

For example, described herein are techniques that use an Lp-PLA2 assay (and particularly an LpPLA2 mass assay) either alone or in combination with another biomarker such as GDF-15, to identify and begin to treat patients for heart failure. These methods and apparatuses (kits, panels, assays) may address many of the shortcomings of known techniques, and in particular may allow early detection of heart failure risk (e.g., NYHA stages I/II) from apparently healthy patients (e.g., patients without previously diagnosed Heart Failure. Also described herein are methods and apparatuses for determining (e.g., using LpPLA2 and one or more additional biomarkers, alone and together), to classify individuals for risk/severity profiles particularly NYHA stage I/II/III/IV of Heart Failure severity.

### SUMMARY OF THE DISCLOSURE

Described herein are new and more accurate diagnostic or prognostic indicators to help identify and stratify individuals having heart failure (HF) or at risk for heart failure, as well as methods for treating such patients. The methods and techniques described herein may be especially useful to detect early stages of congestive heart failure, and may be particularly useful for distinguishing a person having heart failure from a person without heart failure. For example, described herein are biomarkers used alone or in combination for diagnosing or prognosticating and/or treating heart failure. The disclosure also provides methods for preventing further heart failure, treating an existing case of heart failure, or ameliorating the effects from heart failure. These methods may be based on, for example, the diagnosis or prognosis of heart failure by one or more biomarkers.

A biomarker (which is short for “biological marker”) may be a characteristic that is objectively measured and evaluated as an indicator of a normal biological process, a pathogenic process, or pharmacologic response to an intervention. For example, a biomarker may include Lp-PLA₂ (Mass), GDF-15, or sST2.d

In some examples, a single biomarker may be used to perform the methods described herein. In other cases, a combination of HF biomarkers may be chosen. The biomarkers may represent more than one pathophysiologic category, such as myocyte injury/stress, inflammation/oxidative stress, neurohormonal responses to decompensation, extracellular matrix remodeling, and renal dysfunction, may be beneficial, especially if the combination may provide more accurate diagnostic, prognostic, prevention or treatment information regarding the earliest stages of heart failure (namely, NYHA Stages I and II) relative to a healthy patient population. Such orthogonal markers, e.g., markers for two different pathophysiologic categories of a disease syndrome may be utilized to diagnose or prognosticate heart failure. For example, as described herein using two biomarkers, Lp-PLA₂ and GDF-15, together improved diagnostic and prognostic capability for the early stages of Congestive Heart Failure (NYHA class I and II).

Analyzing the measured analyte values by nominal logistic regression, we demonstrate here that two specific biomarkers, Lp-PLA₂^{Mass} (inflammation/oxidative stress) and GDF-15 (myocyte stretch), can be utilized alone or together to provide diagnostic or prognostic value for congestive heart failure, especially at the earliest stages of congestive heart failure. Utilizing a biobank of eighty plasma samples comprising sixty heart failure donor samples and twenty apparently healthy donor samples, the individual analytes Lp-PLA₂^{Mass} and GDF-15 both out-performed the HF disease prediction capabilities of the individual analytes Lp-PLA₂^{Activity}, sST2 and Galectin-3. In addition, the combination of these two analytes (Lp-PLA₂^{Mass}/GDF-15) out-performed the prediction capabilities of any other candidate analyte combination (Lp-PLA₂^{Mass}/sST2 or GDF-15/sST2) and any individual analyte alone. Analyzing the measured analyte values by ordinal logistic regression, we additionally demonstrate here that the combination of these two analytes (Lp-PLA₂^{Mass}/GDF-15) provided excellent specificity and sensitivity (i. e., ROC curves) for discriminating NYHA Heart Failure stage I/II from the healthy donors.

The results demonstrate a clinical cut-point for Lp-PLA₂^{Mass} in heart failure diagnosis or prediction. Cut-points may be chosen to provide any percent of detection, such as greater than 50%, 60%, 70%, 80%, 90%, 91%, 92%, 93%, 94%, 95%, 96%, 97%, 98%, or 99% or a value that is between any two of these values. A value between these values may correspond to a value that is read from a graph that falls between two of the above listed categories. Cut-points may be chosen to provide any level of detection, such as a cut-point that is greater than 200 ng/ml, 300 ng/ml, 400 ng/ml, 500 ng/ml, 600 ng/ml, 700 ng/ml, 800 ng/ml, 1000 ng/ml, 1200 ng/ml, or 1400 ng/ml or a value between any two of these values in a plasma sample. A value between these values may correspond to a value that is read from a graph that falls between two of the above listed categories. A range may have a higher cut-point of less than 300 ng/ml, 400 ng/ml, 500 ng/ml, 600 ng/ml, 700 ng/ml, 800 ng/ml, 1000 ng/ml, 1200 ng/ml, or 1400 ng/ml. The results also demonstrate that a range of values may be useful. A range may have both a lower cut-point and a higher-cut point as listed above. For example, a cut-point may be chosen depending on which other factors are being considered for a diagnosis or prognostication (e.g. risk) (e.g., other test results, other diagnoses, patient symptoms, family history, etc.). Minimal and maximum values of a cut-point may be chosen to assign a diagnosis or risk level; for example to place a sample into a one particular subclass from a range of multiple subclasses. For example, a blood sample with a moderate value of LP-PLA₂^{Mass} (such as from about 400 ng/ml-600 ng/ml) may be used to diagnose an individual with less severe heart failure (placing them in a lower severity heart failure category such as an NYHA Class I class) while a blood sample with a higher LP-PLA₂^{Mass} value (such as from about 600 ng/ml to 800 ng/ml) may be used to diagnose an individual with slightly more severe heart failure (placing them in a higher severity heart failure category such as an NYHA Class II class). In a particular example, the results consistently demonstrate a clinical cut-point (>400 ng/ml) for Lp-PLA₂^{Mass} in heart failure prediction that resides well above the standard clinical cut-point (200 ng/ml) for Lp-PLA₂^{Mass} for prediction of cardiovascular disease (CVD)/stroke.

The results demonstrate a clinical cut-point for GDF-15 in heart failure diagnosis or prediction. Cut-points may be chosen to provide any percent of detection, such as greater than 50%, 60%, 70%, 80%, 90%, 91%, 92%, 93%, 94%, 95%, 96%, 97%, 98%, or 99% or a value that is between any two of these values. A value between these values may correspond to a value that is read from a graph that falls between two of the above listed categories. Cut-points may be chosen to provide any level of detection, such as a cut-point that is greater than 0.5 ng/ml, 0.6 ng/ml, 0.7 ng/ml, 0.8 ng/ml, 0.9 ng/ml, 1.0 ng/ml, 1.1 ng/ml, 1.2 ng/ml, 1.3 ng/ml, 1.4 ng/ml, 1.5 ng/ml, 1.6 ng/ml, 1.7 ng/ml, 1.8 ng/ml, 1.9 ng/ml, or 2.0 ng/ml, or a value between any two of these values in a plasma sample. A value between these values may correspond to a value that is read from a graph that falls between two of the above listed categories. A range may have a higher cut-point of less than 0.5 ng/ml, 0.6 ng/ml, 0.7 ng/ml, 0.8 ng/ml, 0.9 ng/ml, 1.0 ng/ml, 1.1 ng/ml, 1.2 ng/ml, 1.3 ng/ml, 1.4 ng/ml, 1.5 ng/ml, 1.6 ng/ml, 1.7 ng/ml, 1.8 ng/ml, 1.9 ng/ml, or 2.0 ng/ml. The results also demonstrate that a range of values may be useful. A range may have both a lower cut-point and a higher-cut point as listed above. For example, a cut-point may be chosen depending on which other factors are being considered for a diagnosis or prognostication (e.g. risk) (e.g., other test results, other diagnoses, patient symptoms, family history, etc.). Minimal and maximum values of a cut-point may be chosen to assign a diagnosis or risk level; for example to place a sample into a one particular subclass from a range of multiple subclasses. For example, a blood sample with a moderate value of GDF-15 (such as above 1.8 ng/ml and below 2.0 ng/ml) may be used to diagnose an individual with less severe heart failure (placing them in a lower severity heart failure category such as an NYHA Class I class) while a blood sample with a higher GDF-15 value (such as above 2.0 ng/ml) may be used to diagnose an individual with slightly more severe heart failure (placing them in a higher severity heart failure category such as an NYHA Class II class).

For example, described herein are methods of treating heart failure in an individual, the method comprising: determining a level of Lp-PLA2 in a biological sample from the individual (and in some variations one or more additional biomarkers, including two or more biomarkers); scoring a level individual's risk level for heart failure based on the level of Lp-PLA2 determined; and treating the individual with a therapy for heart failure based on the scored level of risk for heart failure.

In particular, the methods described herein may be used when the individual has not previously been diagnosed with heart failure. For example, these methods may be useful in detecting a risk of heart failure (CHF) even at the earliest stages, before the manifestation of significant symptoms (e.g., ACC Stage B, NYHA Class I, NYHA Class II, etc.).

In general, the methods described herein include testing and analyzing the subject's Lp-PLA2 level by testing amount of Lp-PLA2 detectable by immunohistochemistry (e.g., the Lp-PLA2 mass assay) from a sample. Examples of appropriate Lp-PLA2 assays are described in detail herein. Thus, determining a level of Lp-PLA2 in a biological sample from the individual comprises determining a mass level of Lp-PLA2 (e.g. in ng/ml of sample, or any other appropriate units).

As used herein an “individual” may refer to a human or nonhuman subject, which may also be referred to as a patient or subject.

In general, the methods and apparatuses described herein may be configured to score an individual's risk using two or more biomarkers, where one of the biomarkers is the amount of Lp-PLA2 in the sample (e.g., the Lp-PLA2 mass value). In some variations, more than two biomarkers (where one of the biomarkers is Lp-PLA2 (mass)) may be used. As will be described in more detail below, a score to indicate a level individual's risk level for heart failure is generally based on the level of Lp-PLA2, and in some variations, the one or more additional biomarkers used. The score may be determined by any combination of the biomarker values (raw or normalized valued). For example, the score may be determined by a vector value, in which each biomarker has one or more thresholds (or cut points) for determining risk level, and the score may represent a surface or region within the vector space formed by the combined biomarkers. Alternatively or additionally, the score may be determined by adding and/or multiplying and/or dividing (or normalizing) two or more of the biomarkers to generate a value that can be used to assess risk, e.g., by applying one or more thresholds (e.g., cut points).

Described herein are a number of appropriate secondary biomarkers that may be used to determine a score for the level of the individual's risk level for heart failure in combination with the level of Lp-PLA2 determined. As described in greater detail below, some of the possible secondary biomarkers may be much more predictive when used in combination than others. For example, Lp-PLA2 and GDF-15 (or other myocyte stretch markers). Other secondary markers that enhance the predictive value of Lp-PLA2 to detect (in particular, early-stage HF), include: sST2, neurohumoral markers such as mid-regional proadrenomedullin (MR proADM) and endothelin-1), myocyte stress markers (such as mid-regional pro-atrial natriuretic peptide (MR-proANP)), BNP/NTproBNP, and necrotic markers (such as troponin I, troponin T, or troponin I and troponin T, and/or creatine kinase MB (CK-MB)). In addition to the biomarkers described herein, one or more additional indicators may be used to score the individual's risk; these indicator may be used as multiplier or modifiers for the value and/or for the threshold applied to the score. Modifiers may include (but are not limited to): age, gender, diabetes, renal clearance or kidney function/dysfunction (creatine, eGFR, NGAL), adrenal function (ACTH/POMC), past ischemic heart disease, heart function (left ventricular ejection fraction/LVEF), and shortness of breath (active signs and symptoms).

In some variations, the method may include determining both the level of Lp-PLA2 (mass), and scoring may comprise applying one or more cut-points based on at least the Lp-PLA2 mass value. For example, scoring an individual with risk level for heart failure may include scoring the individual having a mass value for Lp-PLA2 between about 400 ng/ml and about 600 ng/ml as having a moderate risk for heart failure; as mentioned above, other cut-point values may be applied (e.g., between about 350 and 500 ng/ml, between about 400 and 550 ng/ml, between above 400 and 625 ng/ml, between about 425 and 625 ng/ml, etc.). An individual having a mass value for Lp-PLA2 above about 600 ng/ml (e.g., above about 550 ng/ml, above about 575 ng/ml, above about 615 ng/ml, above about 625 ng/ml) may be scored as having a high risk for heart failure. As mentioned, in some variations, the risk factor estimation (soring) may be modified by a second biomarker. For example, scoring may include scoring an individual as at risk (e.g., moderate risk) for heart failure when the individual has a level of Lp-PLA2 greater than about 400 ng/ml (or between about 400 ng/ml and 600 ng/ml, etc.) and a level of GDF-15 above about 1.6 ng/mL.

Any of the methods described herein may include an appropriate treatment step. In general, the treatment step includes providing the scored level of risk for heart failure to a health care provider (doctor, nurse, clinic, hospital, etc.) equipped to administer a therapy based on the scored level of risk. Therapies may include advising the patient on activity or behavioral modifications (diet, exercise, etc.) and/or pharmacological therapies (drugs, supplements, etc.) and/or invasive (e.g., surgical) therapies. For example, a treatment may include prescribing one or more pharmacological agents selected from the group consisting of: angiotensin-converting enzyme (ACE) inhibitors, angiotensin-receptor blockers (ARBs), Beta blockers, diuretics, aldosterone blockers, digitalis, hydralazine and nitrates, statins, aspirin and warfarin. Behavioral modifications and/or collaborative care, particularly in patients with risk (moderate to high) for HF based on the assays described herein, but which are not presenting with frank HF, may be highly effectively. For example, such patients may be treated by collaborative care (e.g., cardiology consults, echocardiology, and coaching) to reduce risk for future HF.

For example, described herein are methods of treating heart failure in an individual, the method comprising: determining a level of LP-PLA2 and a second biomarker in a biological sample from the individual; scoring the level individual's risk level for heart failure based on the level of Lp-PLA2 and the level of the second biomarker determined; and treating the individual with a therapy for heart failure based on the scored level of risk for heart failure.

For example, a method of treating heart failure in an individual that has not previously been diagnosed with heart failure may include: determining a level of LP-PLA2(Mass) and a level of GDF-15 in a biological sample from the individual; scoring the level individual's risk level for heart failure based on the level of Lp-PLA2 and the level of the second biomarker determined; and treating the individual with a therapy for heart failure based on the scored level of risk for heart failure.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows the details of the samples that were used for performing the analysis described herein, including a first set of Biobank blood plasma samples with an NYHA classification of healthy and Classes 1-4.

FIG. 2 continues the details shown of FIG. 1.

FIGS. 3A-3B show the results of biomarker testing (for Lp-PLA2 mass, Lp-PLA2 activity, GDF-15 mass, sST2, and Galectin 3) as described herein on the samples shown in FIGS. 1 and 2.

FIG. 4 shows an analysis of the Lp-PLA2 mass assay performed on the healthy and NYHA class 1 and 2 samples described in FIGS. 1 and 2, including a nominal regression.

FIG. 5 shows an analysis of the Lp-PLA2 activity assay performed on the healthy and NYHA class 1 and 2 samples described in FIGS. 1 and 2, including a nominal regression.

FIG. 6 shows an analysis of the GDF-15 mass assay performed on the healthy and NYHA class 1 and 2 samples described in FIGS. 1 and 2, including a nominal regression.

FIG. 7 shows an analysis of the sST2 assay performed on the healthy and NYHA class 1 and 2 samples described in FIGS. 1 and 2, including a nominal regression.

FIG. 8 shows an analysis of the Galectin-3 mass assay performed on the healthy and NYHA class 1 and 2 samples described in FIGS. 1 and 2, including a nominal regression.

FIG. 9 shows an analysis of the combined Lp-PLA2 mass and GDF-15 assays performed on the healthy and NYHA class 1 and 2 samples described in FIGS. 1 and 2, including a nominal regression.

FIG. 10 shows an analysis of the combined Lp-PLA2 mass and sST2 assays performed on the healthy and NYHA class 1 and 2 samples described in FIGS. 1 and 2, including a nominal regression.

FIG. 11 shows an analysis of the combined GDF-15 and sST2 assays performed on the healthy and NYHA class 1 and 2 samples described in FIGS. 1 and 2, including a nominal regression.

FIG. 12 (Table 2) summarizes a statistical analysis from the subset of Biobank blood plasma samples from healthy and Classes 1 and 2 donors across different biomarkers and combinations of biomarkers.

FIG. 13 (Table 3) shows how the heart failure biomarkers tested as described herein classified the subset of samples from the subset of healthy and Classes 1 and 2 donors shown in FIGS. 1, 2 and 3A-B.

FIG. 14 shows an analysis of the Lp-PLA2 mass assay performed on the healthy and NYHA classes 1, 2, 3 and 4 samples described in FIGS. 1 and 2, including a nominal regression.

FIG. 15 shows an analysis of the Lp-PLA2 activity assay performed on the healthy and NYHA classes 1, 2, 3 and 4 samples described in FIGS. 1 and 2, including a nominal regression.

FIG. 16 shows an analysis of the GDF-15 mass assay performed on the healthy and NYHA classes 1, 2, 3 and 4 samples described in FIGS. 1 and 2, including a nominal regression.

FIG. 17 shows an analysis of the sST2 assay performed on the healthy and NYHA classes 1, 2, 3 and 4 samples described in FIGS. 1 and 2, including a nominal regression.

FIG. 18 shows an analysis of the Galectin-3 mass assay performed on the healthy and NYHA classes 1, 2, 3 and 4 samples described in FIGS. 1 and 2, including a nominal regression.

FIG. 19 shows an analysis of the Lp-PLA2 mass and GDF-15 assays performed on the healthy and NYHA classes 1, 2, 3 and 4 samples described in FIGS. 1 and 2, including a nominal regression.

FIG. 20 shows an analysis of the Lp-PLA2 mass and sST2 assays performed on the healthy and NYHA classes 1, 2, 3 and 4 samples described in FIGS. 1 and 2, including a nominal regression.

FIG. 21 shows an analysis of the GDF-15 and sST2 assays performed on the healthy and NYHA classes 1, 2, 3 and 4 samples described in FIGS. 1 and 2, including a nominal regression.

FIG. 22 (Table 4) shows a summary of the statistical analysis of blood plasma samples shown in FIGS. 1, 2 and 3A-B analyzed with biomarkers alone and in combination. The analysis includes samples with an NYHA classification of healthy or class 1-4.

FIG. 23 (Table 5) shows how the heart failure biomarkers tested as described herein classified the samples from the healthy and Classes 1-4 donors shown in FIGS. 1, 2 and 3A-B.

FIG. 24 (Table 6) shows a summary and comparison of how the heart failure biomarkers tested as described herein on the samples shown in FIGS. 1, 2 and 3A-B classified the NYHA classes 1 and 2 alone for heart failure, compared with how they classified NYHA classes 1-4 together for heart failure.

FIG. 25 shows an ordinal regression analysis of the Lp-PLA2 mass and GDF-15 biomarkers on the samples shown in FIGS. 1, 2 and 3A-B classified as the NYHA classes 1 and 2.

FIG. 26 shows an ordinal regression analysis of the Lp-PLA2 mass and GDF-15 biomarkers on the samples shown in FIGS. 1, 2 and 3A-B classified as the NYHA classes 1-4.

FIG. 27 shows a comparison of heart failure (CHF) versus healthy individuals for various biomarkers and combinations of biomarkers described herein, using a 40-patient sample with 29 CHF and 11 healthy donors.

FIG. 28 shows a comparison of NYHA class 2 versus healthy individuals for various biomarkers and combinations of biomarkers described herein, using a 40-patient sample with 29 CHF and 11 healthy donors.

FIG. 29 shows a comparison of NYHA class 3 versus healthy individuals for various biomarkers and combinations of biomarkers described herein, using a 40-patient sample with 29 CHF and 11 healthy donors.

FIG. 30 shows a comparison of NYHA class 1 and 2 versus healthy individuals for various biomarkers and combinations of biomarkers described herein, using a 40-patient sample with 29 CHF and 11 healthy donors.

FIG. 31 shows a comparison of NYHA class 2 and 3 versus healthy individuals for various biomarkers and combinations of biomarkers described herein, using a 40-patient sample with 29 CHF and 11 healthy donors.

FIG. 32 shows a comparison of NYHA class 3 and 4 versus healthy individuals for various biomarkers and combinations of biomarkers described herein, using a 40-patient sample with 29 CHF and 11 healthy donors.

FIG. 33 shows a comparison of NYHA code groups versus healthy individuals for various biomarkers described herein (Lp-PLA2 mass on the left, GDF-15 on the right), using a 40-patient sample with 29 CHF and 11 healthy donors.

FIG. 34 is a comparison of CHF versus healthy (combined assays), as a bivariate fit of Lp-PLA2 mass by GDF-15 mass.

FIG. 35 is a comparison of heart failure (CHF) versus healthy (combined assays) showing a prediction profiler of Lp-PLA2 mass and GDF-15 assays.

FIG. 36 is a comparison of NYHA code versus healthy (combined assays) showing a prediction profiler of Lp-PLA2 mass and GDF-15 assays.

### DETAILED DESCRIPTION

Described herein are methods and apparatuses (panels, assays, etc.) using one or more diagnostic biomarker, typically including Lp-PLA2, for identifying and stratifying risks of heart failure (HF), and especially for identifying and stratifying early stages of congestive heart failure, and treating individuals based on these. Also described are prognostic biomarker indicators (methods and apparatuses) that may identify future risk of heart failure or heart failure related events and methods for treating an individual based on these indicator assays. Such biomarker indicators may be more accurate than those provided by currently available markers. Such indicators may be used alone or with other indicators described herein or may be used in conjunction with existing biomarkers. The disclosure also provides methods for addressing heart failure, such as preventing further heart failure, treating an existing case of heart failure, or ameliorating effects from heart failure. These methods for addressing heart failure may be based on, for example, the diagnosis or prognosis of heart failure by one or more biomarkers.

Initially, candidate HF biomarkers, including Lp-PLA₂^{Mass}, Lp-PLA2^{Activity}, GDF-15, sST2 and Galectin-3, were chosen for analysis. Lp-PLA₂ (lipoprotein-associated phospholipase A₂) is an enzyme found in the blood. It is associated with low-density lipoprotein in the blood and is correlated with the development of atherosclerosis, coronary heart disease, inflammation, and stroke. However, it is not known what specific role it might play in the progression or prevention of any of these diseases or how its role might be different under different circumstances For example, it is not known if Lp-PLA₂ plays a role in causing such diseases, preventing damage from such diseases, or has another role. The total mass amount of Lp-PLA₂ can be measured (such as using as an Lp-PLA₂^{Mass} assay) or the activity level of the Lp-PLA₂ enzyme can be measured (using an Lp-PLA₂^{Activity} assay); these assays measure different qualities of the Lp-PLA₂. They may give different results that do not directly correspond with each other. GDF-15 (Growth Differentiation Factor-15) is a protein of the transforming growth factor beta (TGF-β) superfamily that regulates tissue differentiation and maintenance and is expressed in blood and cancer cells. Galectin-3 is a carbohydrate binding protein that plays a role in various cell processes, including apoptosis, immunity, cell adhesion and T-cell regulation. Additional biomarkers that may be used, particularly in combination with another biomarker, and particularly the Lp-PLA2 (mass) marker, include neurohumoral markers (such as mid-regional proadrenomedullin (MR proADM), and endothelin-1), analytes of myocyte stress (e.g., atrial natriuretic peptide, Mid-regional pro-atrial natriuretic peptide (MR-proANP)), and necrotic markers (e.g., troponin I/T, CKMB).

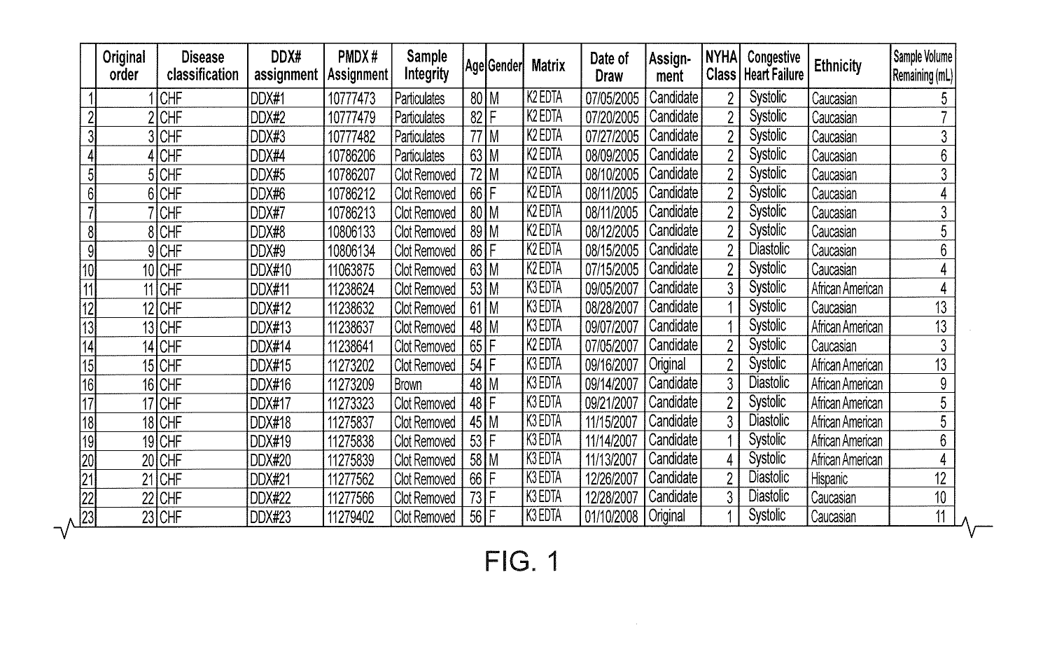

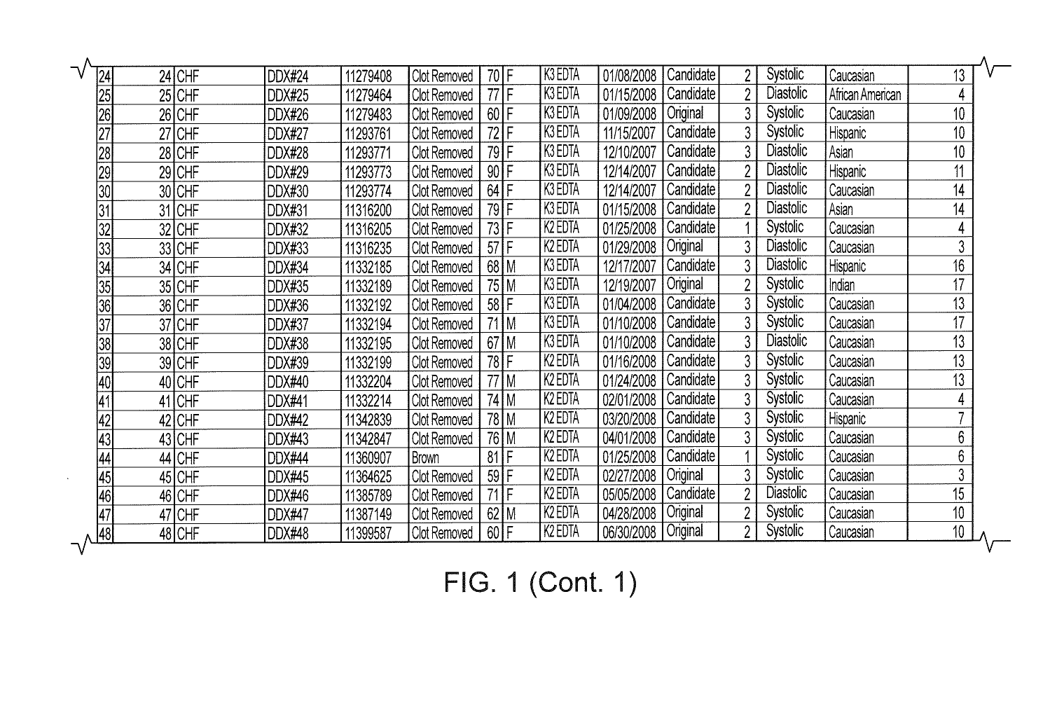

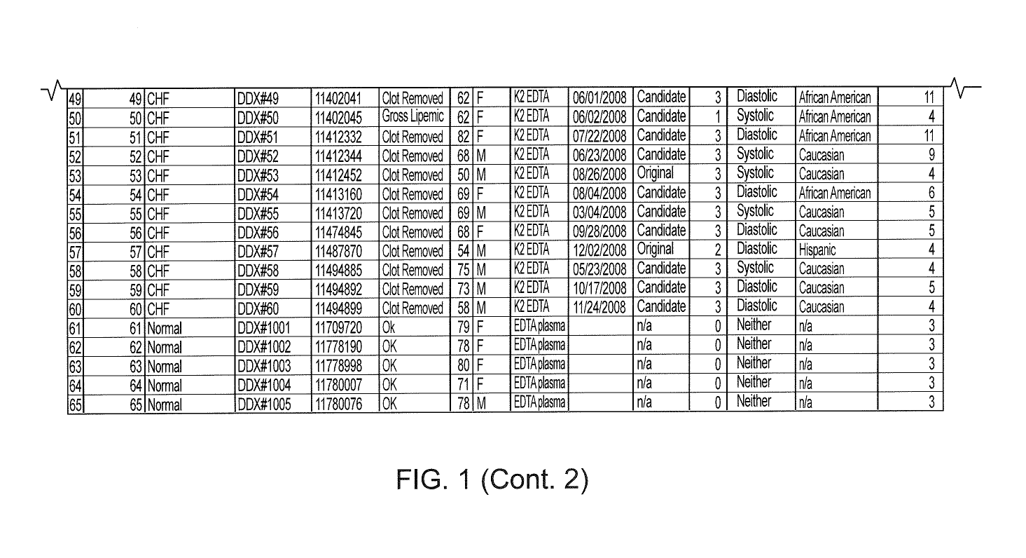

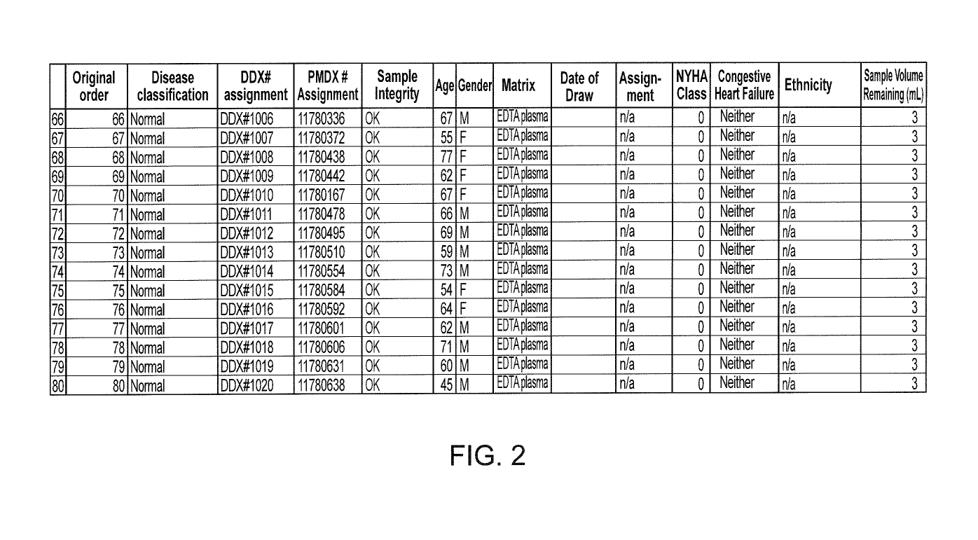

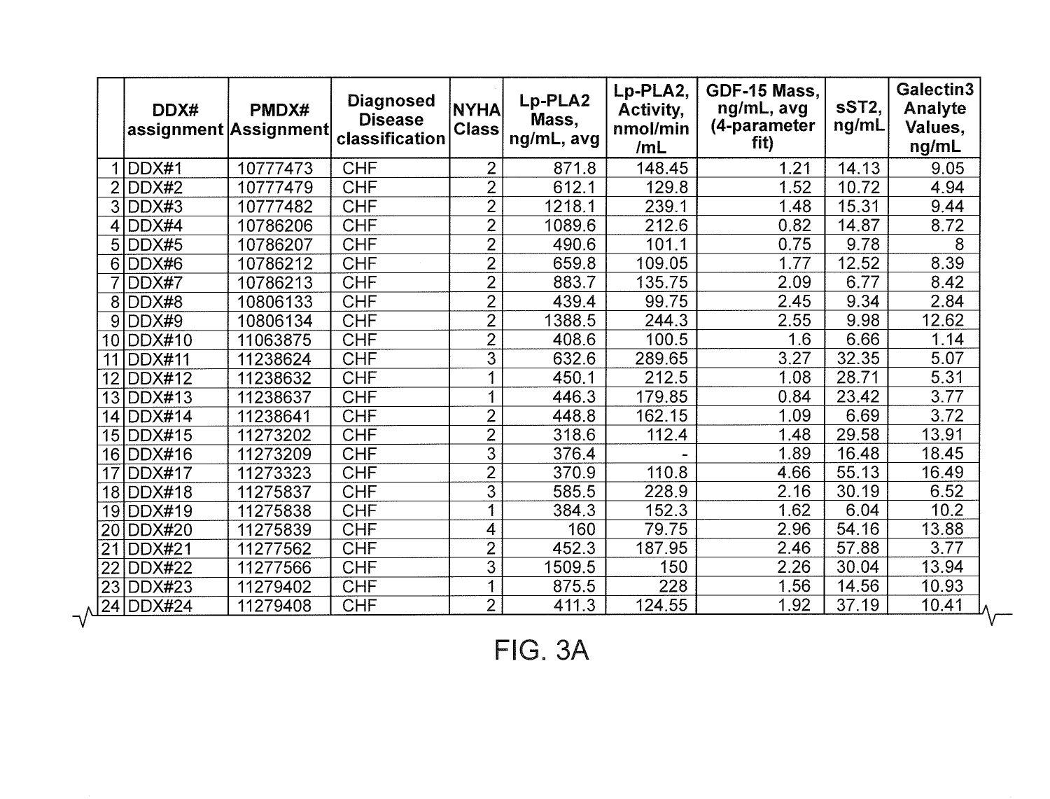

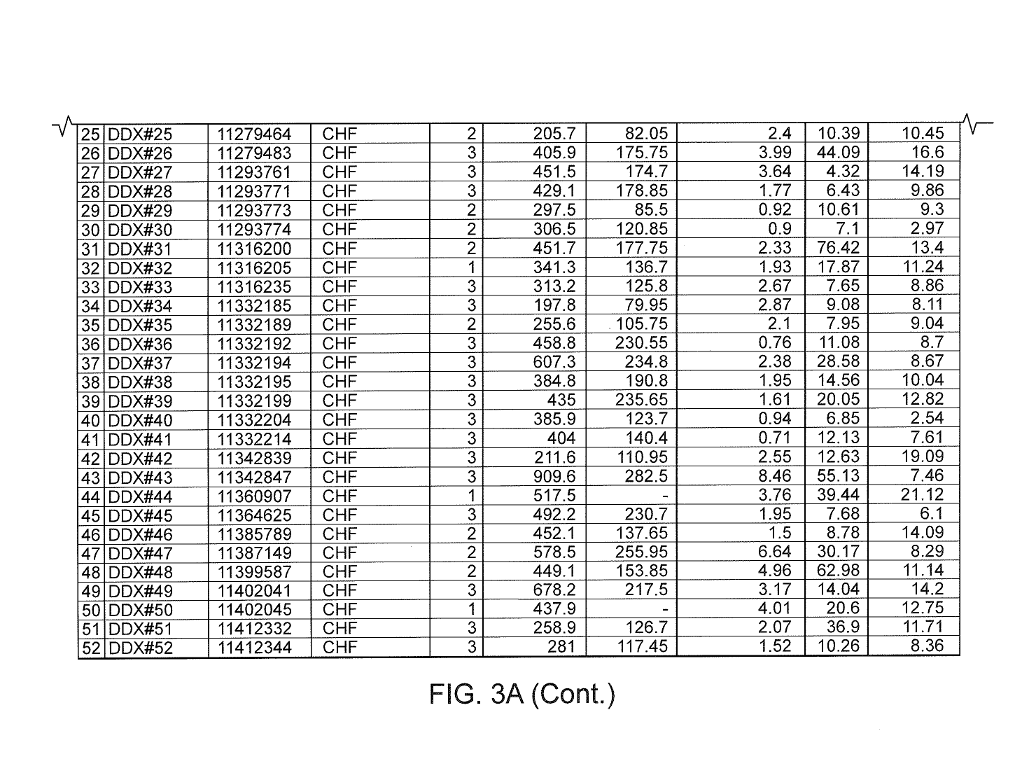

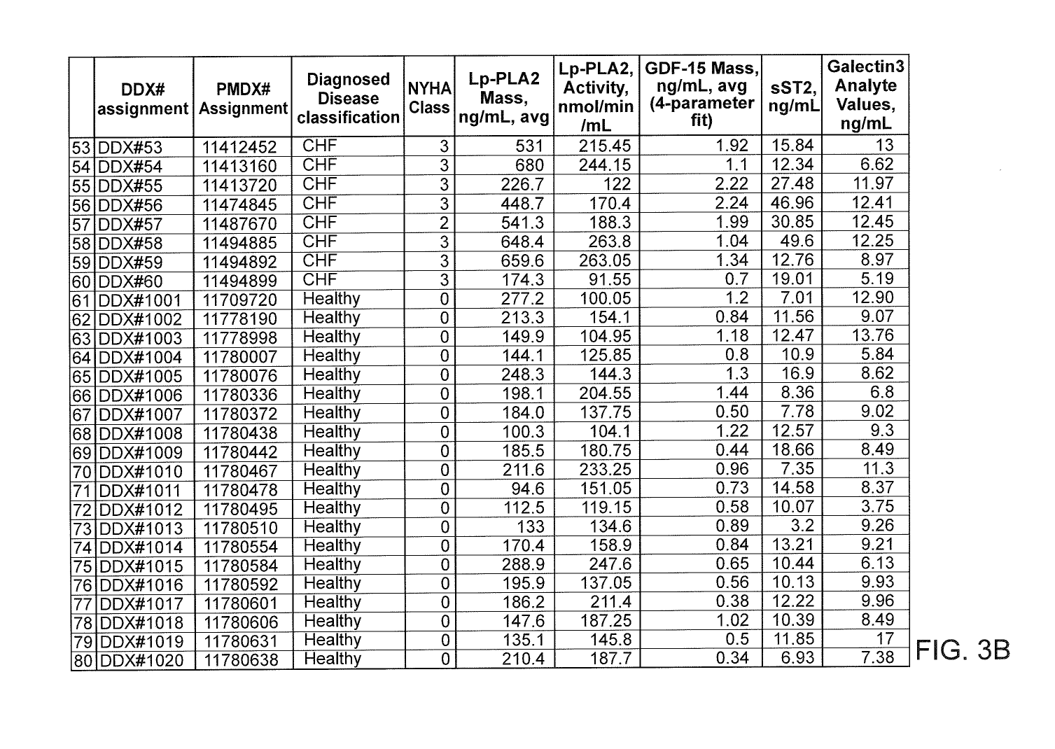

As will be described herein, analyte levels of candidate HF biomarkers were analyzed on blood plasma from a first group of healthy individuals and individuals with heart failure. A BioBank of patient samples was assembled which had two age distribution-matched cohorts of EDTA-plasma samples: sixty Congestive Heart Failure donor samples of various NYHA classes (1/2/3/4) and twenty apparently healthy donor samples as described in FIGS. 1 and 2. Other socio-demographic factors, including ethnicity and sex, were taken into account in the selection of the sixty CHF plasma samples to reflect the United States population. Analyte levels for a first set of candidate HF biomarkers (Lp-PLA₂^{Mass}, Lp-PLA₂^{Activity}, GDF-15, sST2 and Galectin-3) were screened using a commercial IVD/RUO (in vitro diagnostic/research use only) ELISA or enzyme activity assays. The results of the testing are shown in FIGS. 3A-B.

For example, for LP-PLA2 (mass) assays (PLAC Test, mass assay, diaDexus Inc., South San Francisco, Calif.) were performed according to published protocols. Samples (e.g., 1-40 μl) of each sample were applied onto the assay plate wells and the plate was incubated for 10 minutes at room temperature. Two hundred micro liters of the anti-rLp-PLA₂ antibody-HRP conjugate solution were added to each well and the plate was incubated at room temperature for 3 hr without sealing. The plate was then washed with TBS/T buffer for 4 times and incubated with 100 μl of TMB substrate solution for 20 minutes at the room temperature in dark. The reaction was stopped by adding 100 μl of 1 M HCl each well and concentrations were determined by reading of the plate (e.g., in a SPECTRAmax M5 plate reader at 450 nm).

For the LP-PLA2 (activity) assay (PLAC test for activity, diaDexus Inc., South San Francisco, Calif.), activity was determined using an enzyme assay on an automated clinical chemistry analyzer (e.g., Olympus AU 2700) following the manufacturer's directions. Recombinant Lp-PLA₂ (rLp-PLA₂) enzyme kinetic assays in the study were carried out by using the CAM assay kit (diaDexus, Inc.). Basically, in a 96-well plate, reactions were started by adding 110-134 μl of the reaction buffer to each well containing 1-25 μl of Lp-PLA₂ samples according to the protocol by the manufacturer. The volumes of enzyme and reaction buffer were depended on the individual experiment. The reactions were followed at OD405 nm (absorbance) in a reader (e.g., SPECTRAmax M5 plate reader) and the steady state reaction rates of the first 3 or 5 minutes depending on the experiments were averaged.

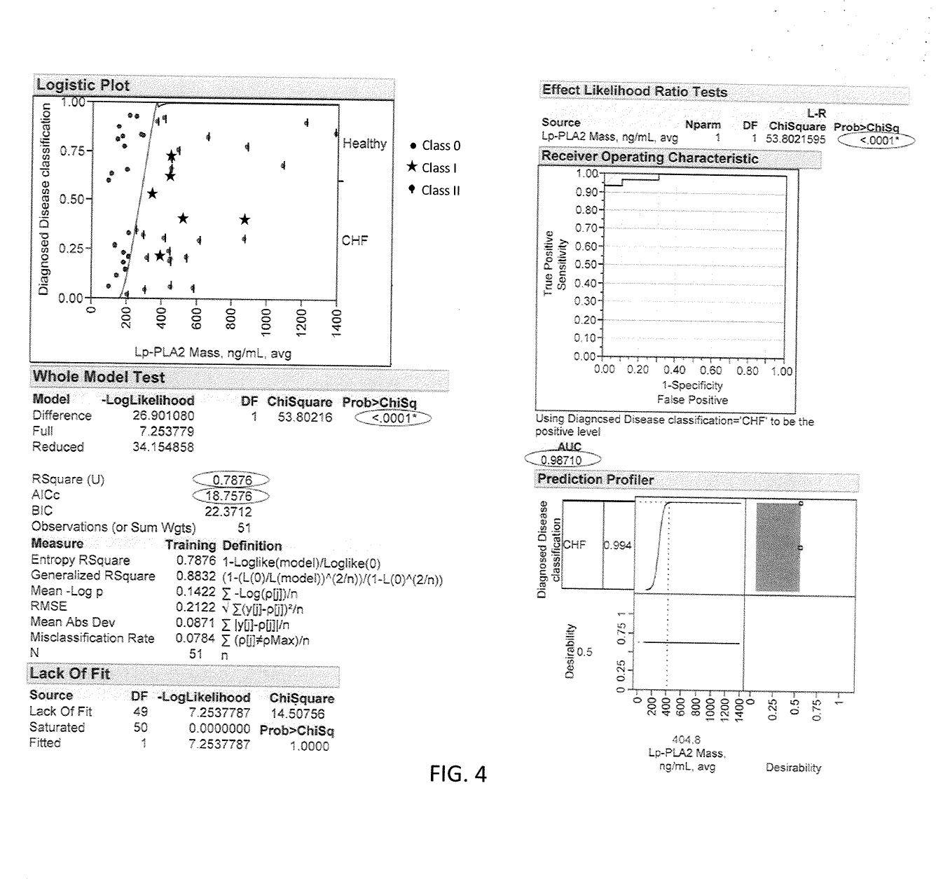

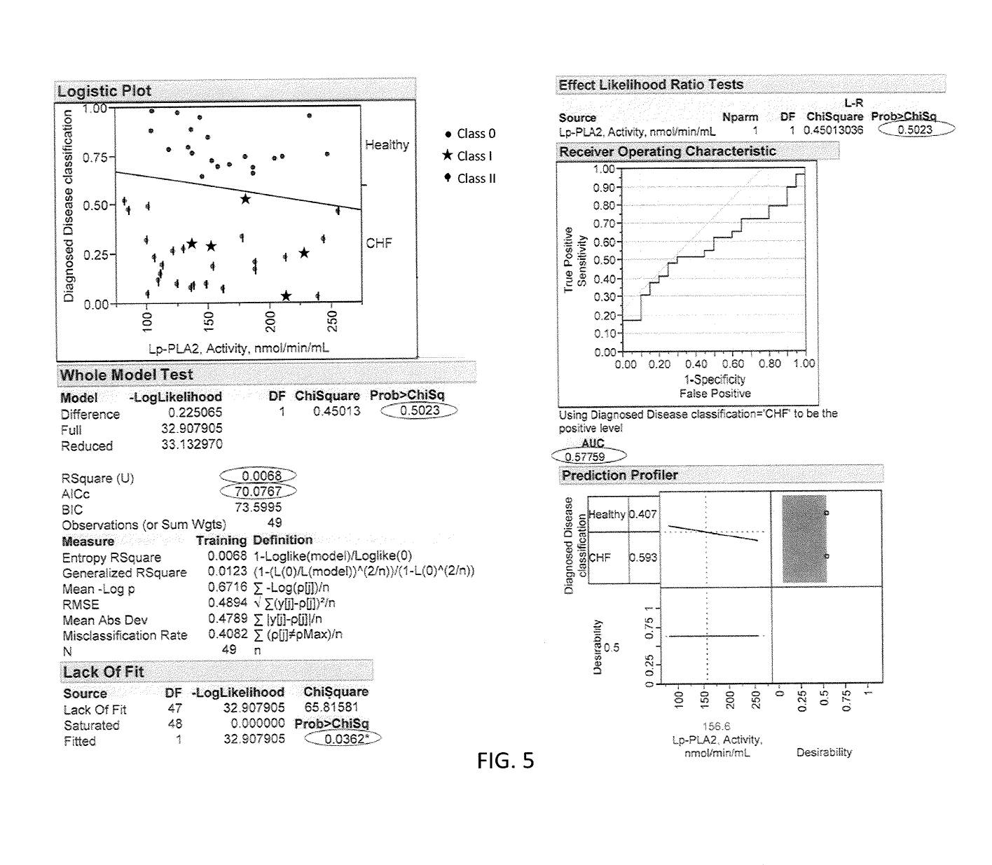

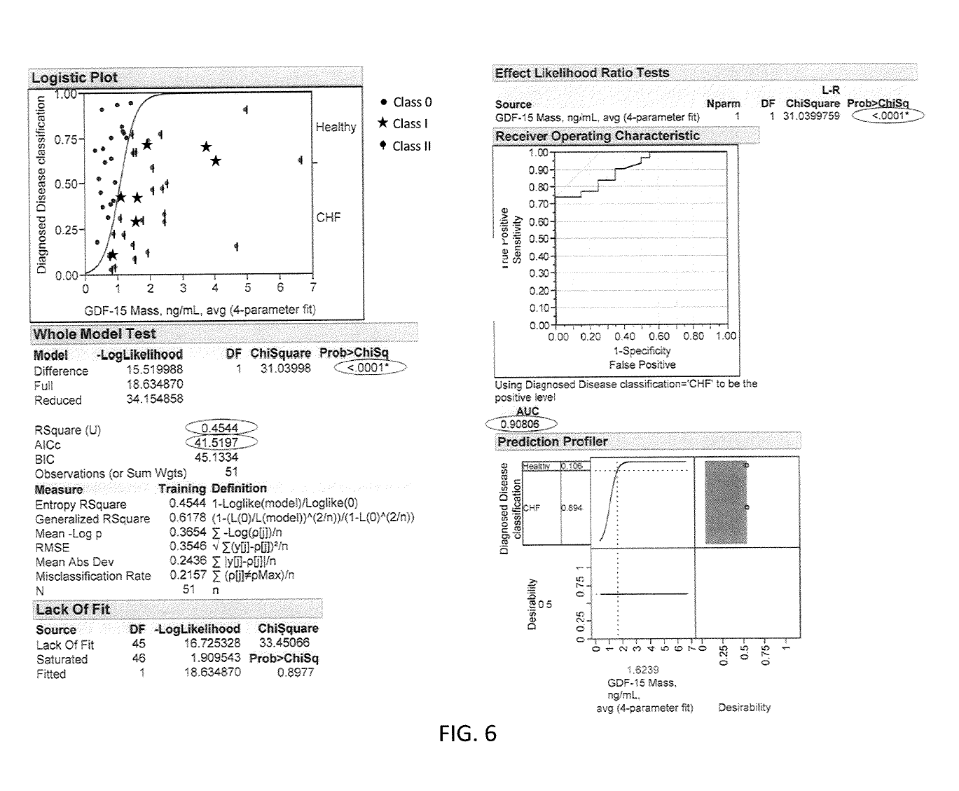

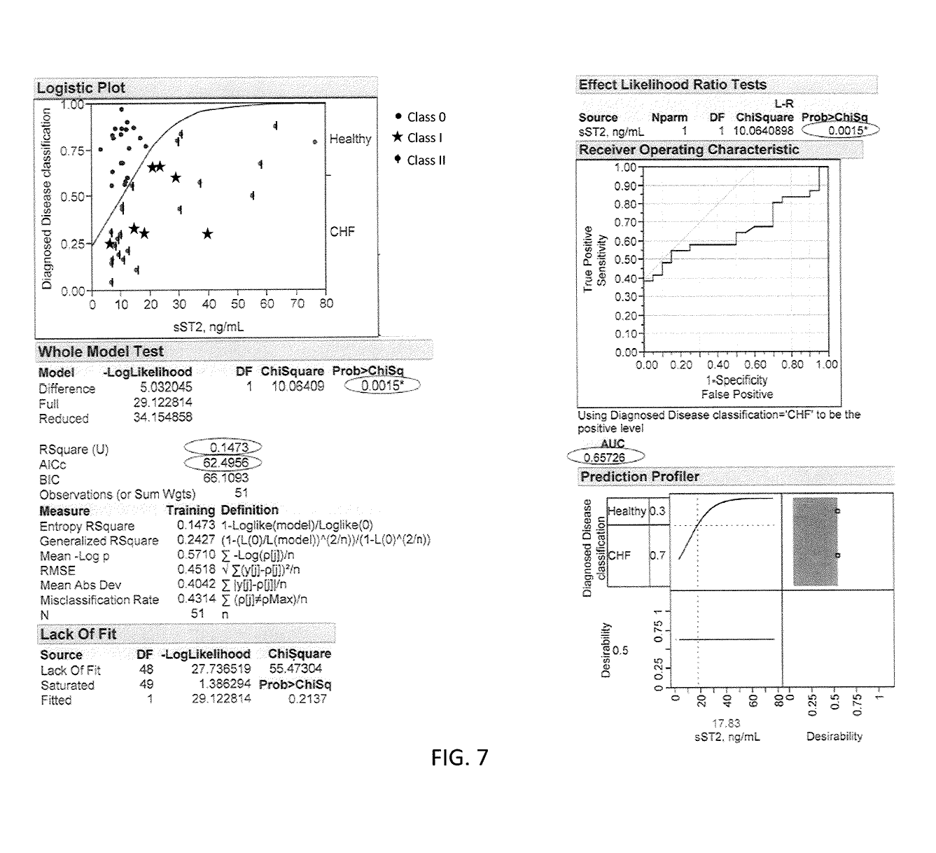

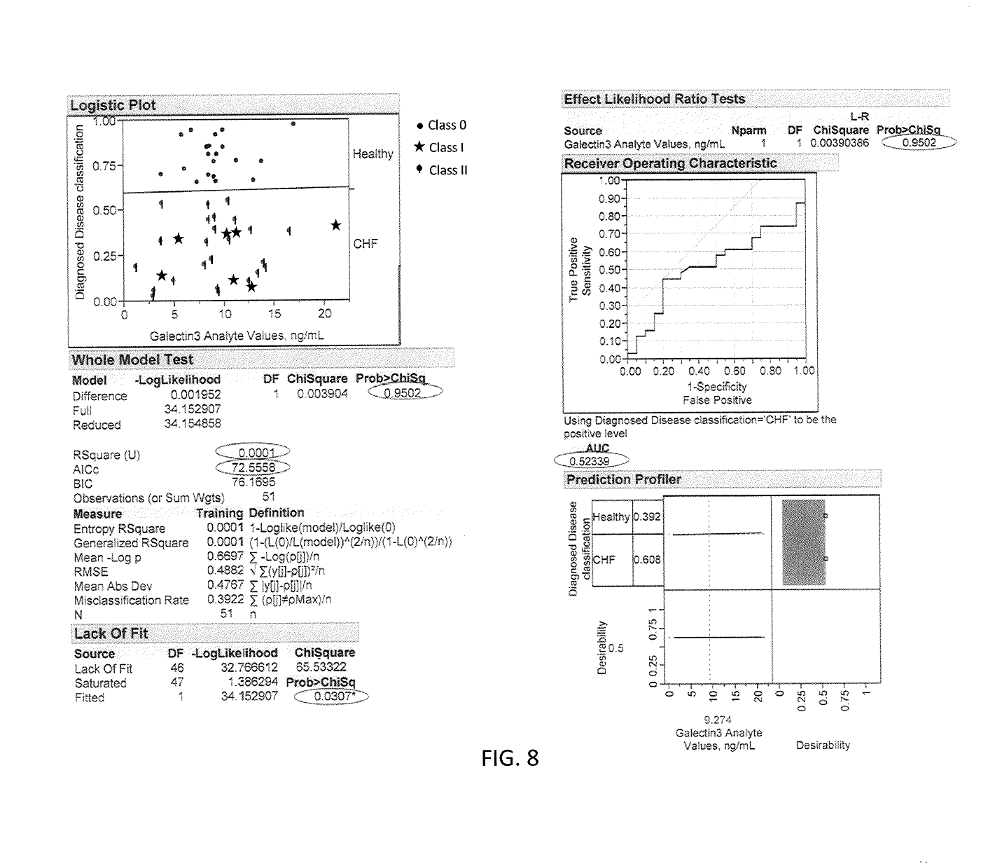

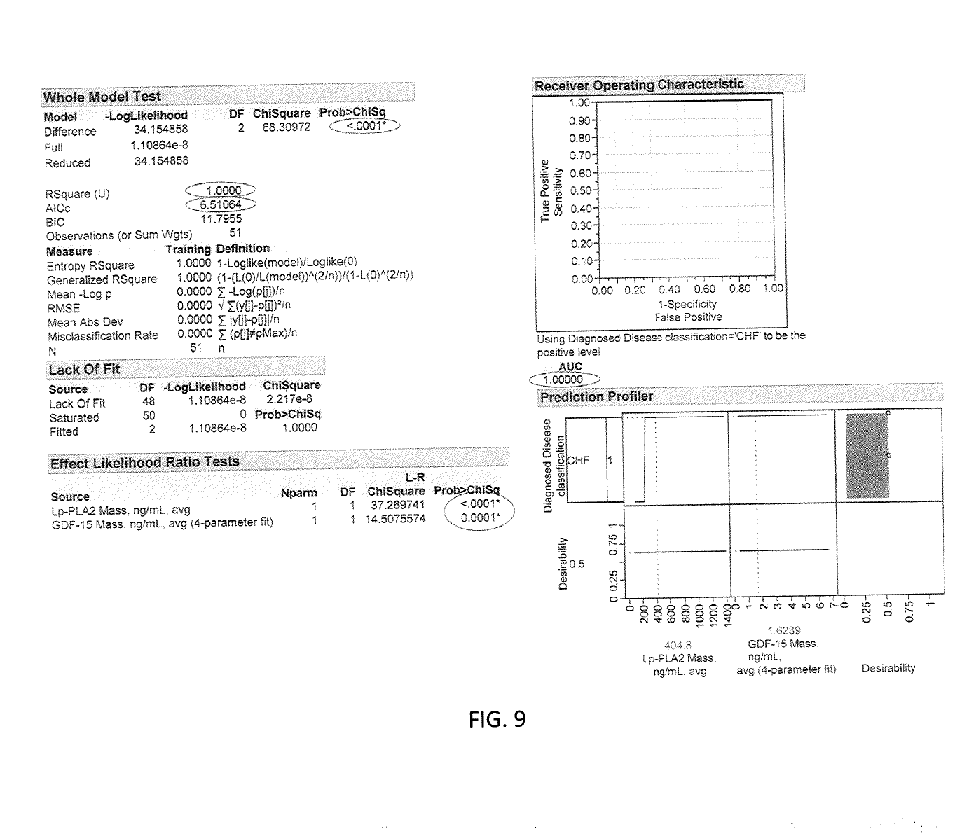

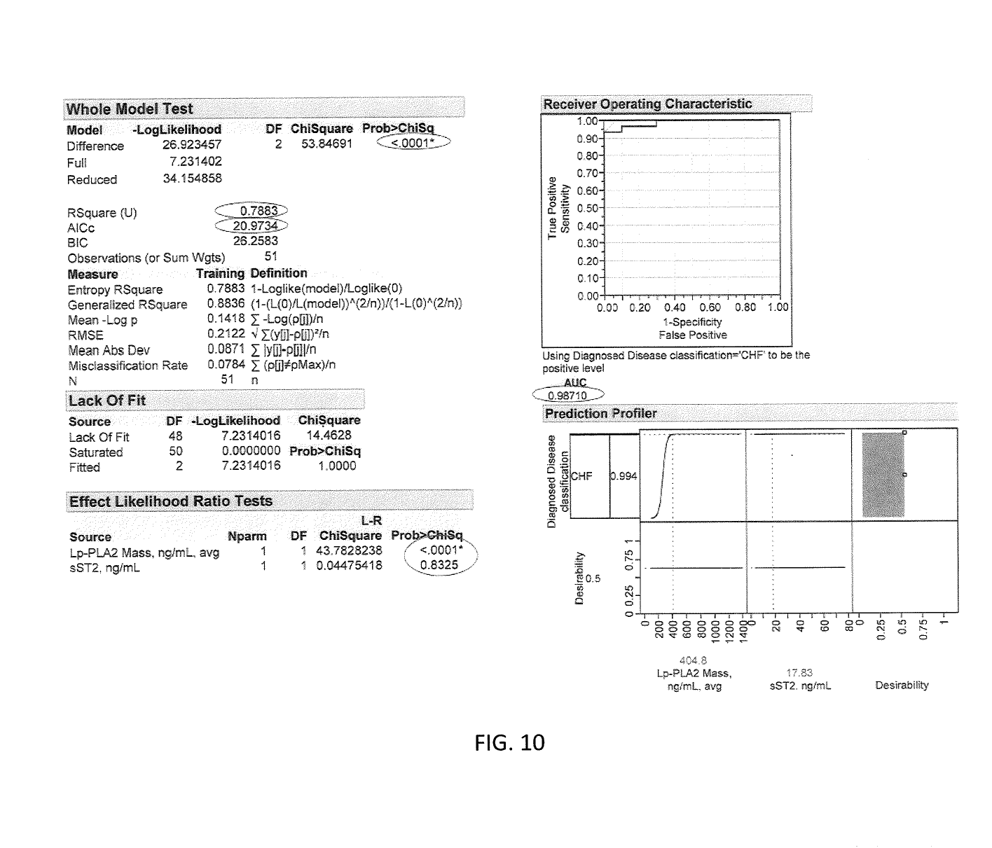

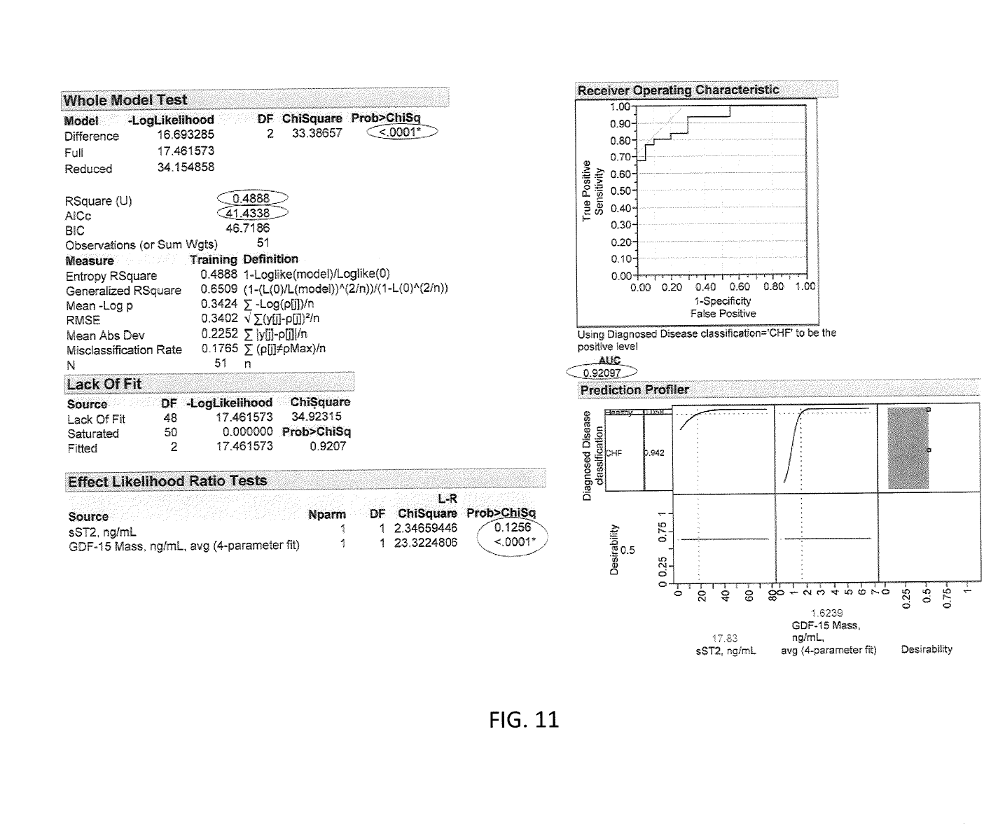

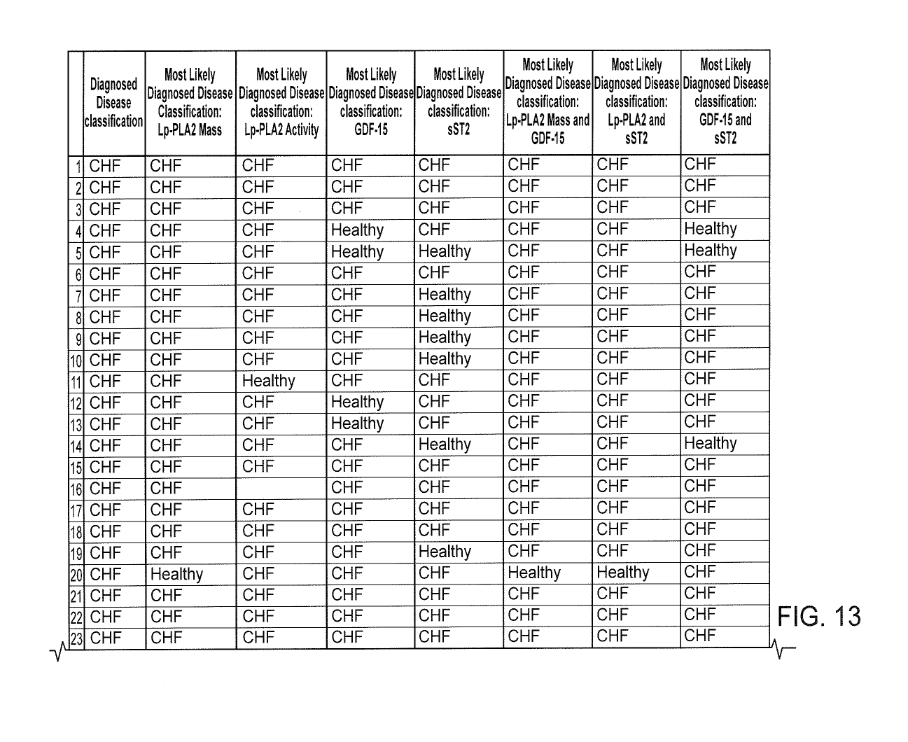

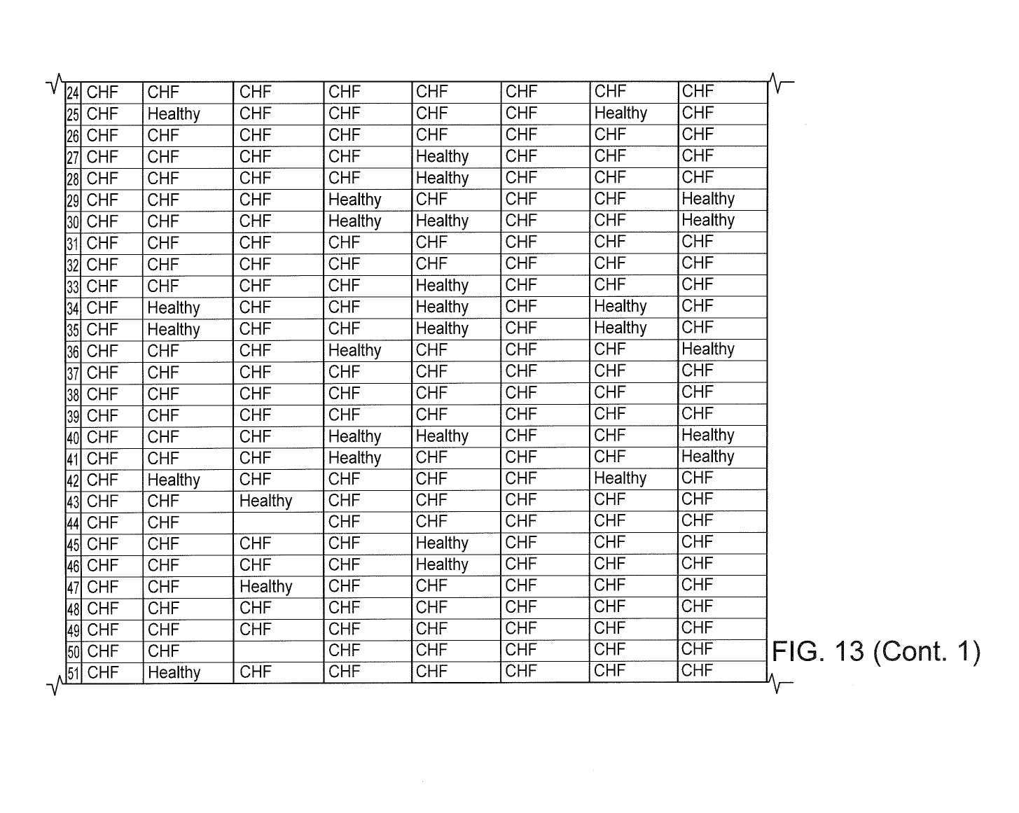

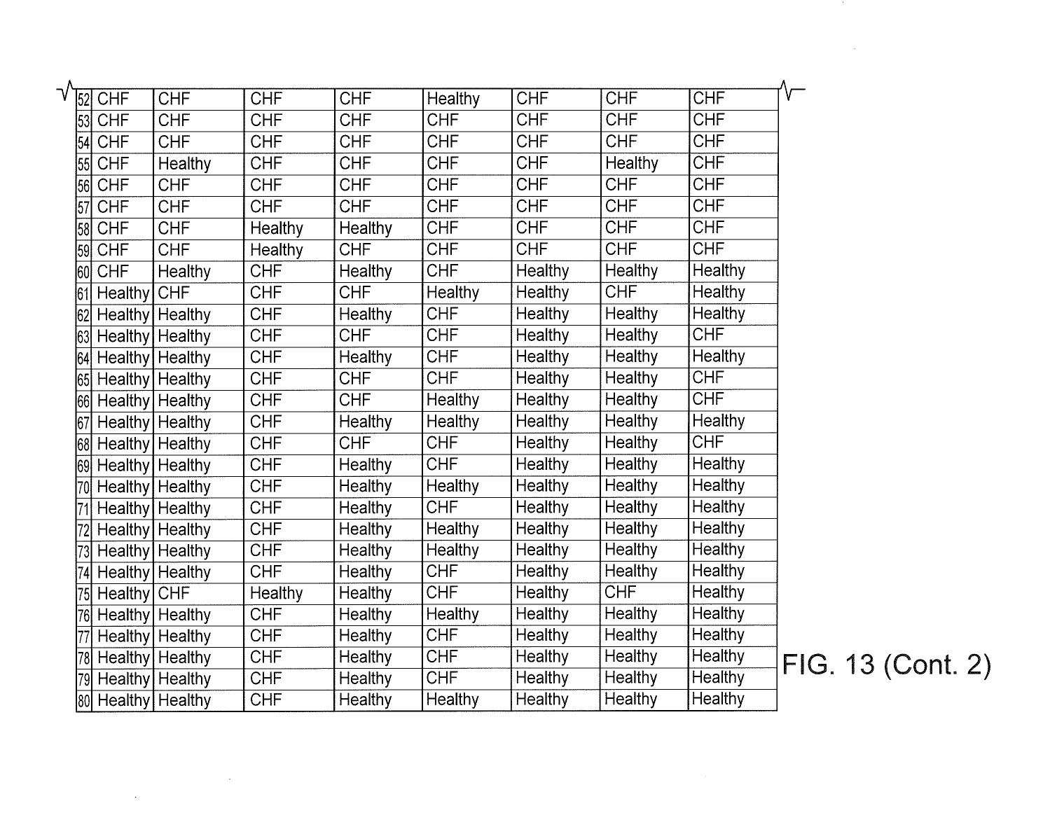

The measured analyte values were analyzed by nominal logistic regression and ordinal logistic regression using a commercially available statistical software package (JMP 10.0.2). The statistical analyses of the individual and combined assays are shown in FIGS. 4-11. The data was modeled using either a single analyte or a combination of two analytes relative to the apparently healthy donor samples for, (a), the NYHA class 1/2 samples, or, (b), the entire battery of NYHA classes 1/2/3/4. The NYHA classes 1/2 represent the early stages of heart failure whereas the NYHA classes 3/4 represent the later stages of heart failure. The nominal response models were evaluated by several established statistical criteria including “whole model p-value”, R-squared (U), AICc, and the Effect Likelihood Ratio Tests of individual analytes. The software was also utilized to generate ROC (receiver operating characteristic) including an area-under-curve calculation as well as a prediction formula giving the most likely response (healthy or CHF) from the nominal response model. The ROC curve is a graphical representation of the relationship between false-positive (specificity) and true-positive rates (sensitivity) with the goal of maximizing the area under the curve (AUC). The effect on the AUC values (area under the ROC curve) by the inclusion of individual analytes, or combinations of analytes, in the model were evaluated (i.e., high AUC values indicate that the model has good predictive ability). The prediction formula functionality was used to pick the most likely level of each row (healthy or CHF) based on the computed probabilities, as shown in FIG. 13. A tally of the number of correctly predicted disease states was scored relative to the known qualities of the donor sample. Finally, an ordinal logistic regression was modeled using the individual NYHA class levels for the combination of Lp-PLA₂^{Mass} and GDF-15 to determine ROC curves for the individual classes of NYHA heart failure.

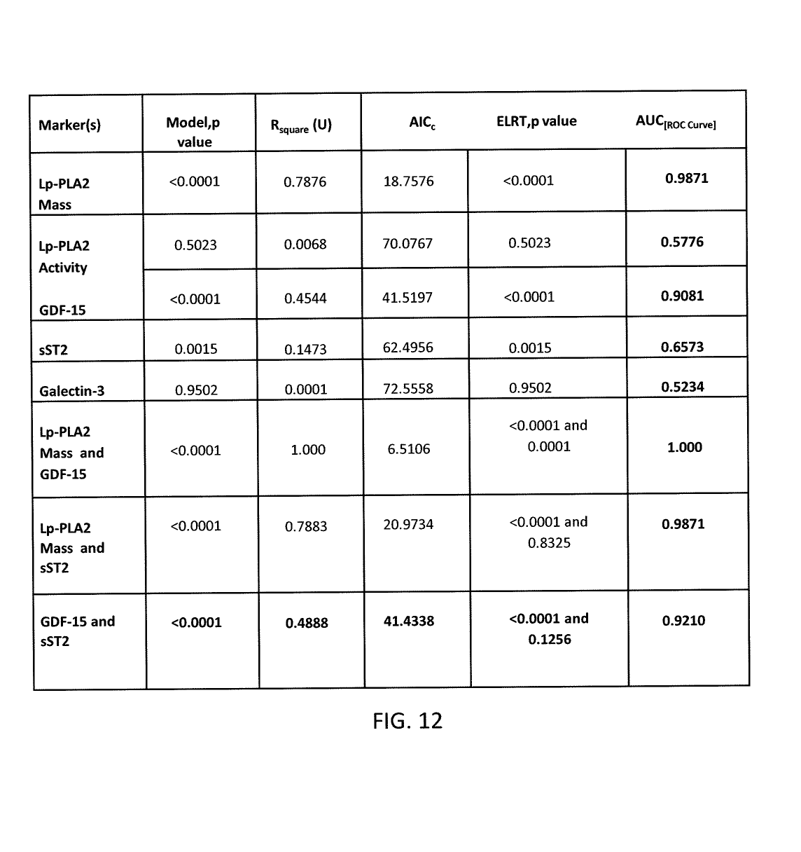

Analyte levels of candidate HF biomarkers analyzed on blood plasma from the first Biobank sample having healthy individuals and individuals with heart failure was further performed as follows. The nominal logistic regression was first performed on a subset of fifty-one samples comprising 31 CHF samples comprising NYHA class 1/2 with 20 healthy samples as control (FIG. 12 (Table 2)). Lp-PLA₂^{Mass} assay, but not Lp-PLA₂^{Activity} assay, gave excellent modeling results with an R_{squared} of 0.7876 and AUC_{ROC Curve} of 0.9871 for the ELISA version compared to R_{squared} of 0.0068 and AUC_{ROC Curve} 0.5776 for the enzymatic version. The analyte GDF-15 also gave good modeling results with an R_{squared} of 0.4544 and AUC_{ROC Curve} of 0.9081 compared to the other two analytes, sST2 (0.1473 and 0.6573, respectively) and Galectin-3 (0.0001 and 0.5234, respectively). The prediction formula for the Lp-PLA₂^{Mass} correctly predicted 70/80 donor sample's disease state. Individually, the Lp-PLA₂^{Mass} assay provided the most accurate prognostic information regarding the earliest stages of heart failure (namely, NYHA Stages I and II) among the individual analytes studied here. Furthermore, the Lp-PLA₂^{Mass} assay in combination with GDF-15 gave even more impressive results: an R_{squared} of 1.000 and an AUC_{ROC Curve} of 1.000 with excellent Effect Likelihood Ratio Test p-values of 0.0001 (or better) for the two analytes together. This combination of analytes overall gave superior results to the combination of Lp-PLA₂^{Mass}/sST2 or GDF-15/sST2. The prediction formula for the combination of Lp-PLA₂^{Mass} and GDF-15 combination correctly predicted 78/80 donor sample's disease state (FIG. 13/Table 3). These results strongly suggest that the combination of the two analytes, Lp-PLA₂^{Mass} and GDF-15 provide accurate prognostic or diagnostic information regarding the earliest stages of heart failure (namely, NYHA Stages I and II). In a particular example, the combination of the two analytes, Lp-PLA₂Mass (cut-off 400 ng/mL) and GDF-15 (cut-off 1.6 ng/mL), provide accurate prognostic or diagnostic information regarding the earliest stages of heart failure (namely, NYHA Stages I and II).

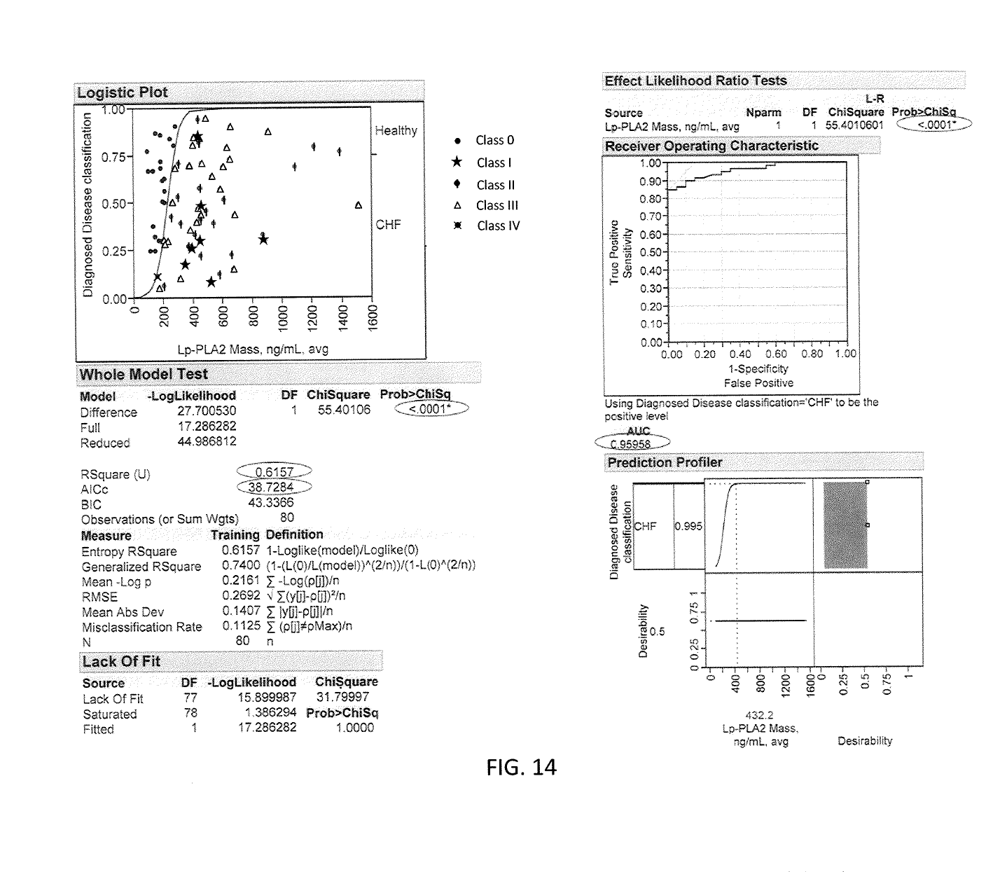

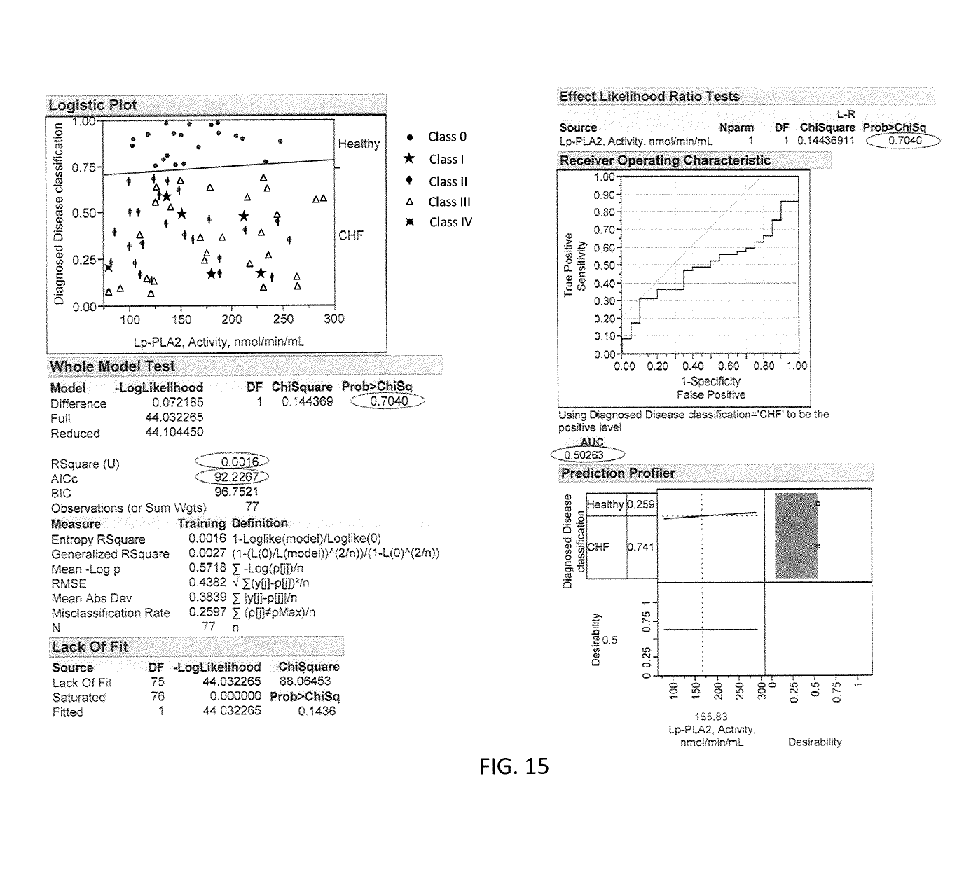

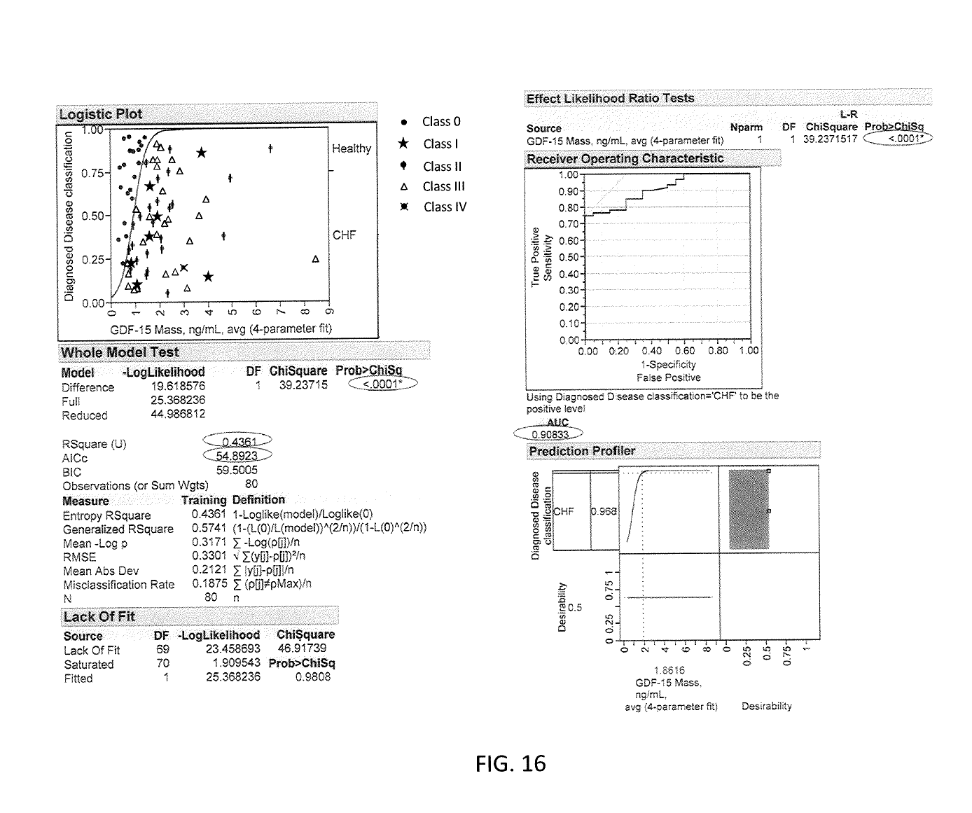

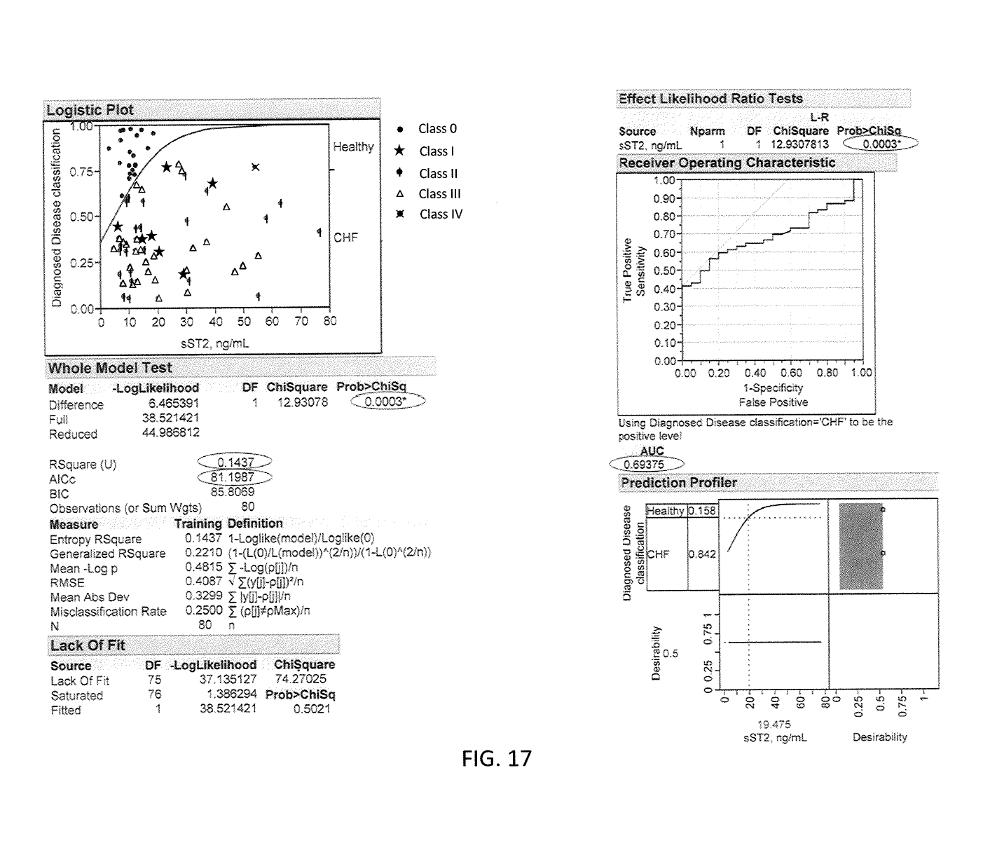

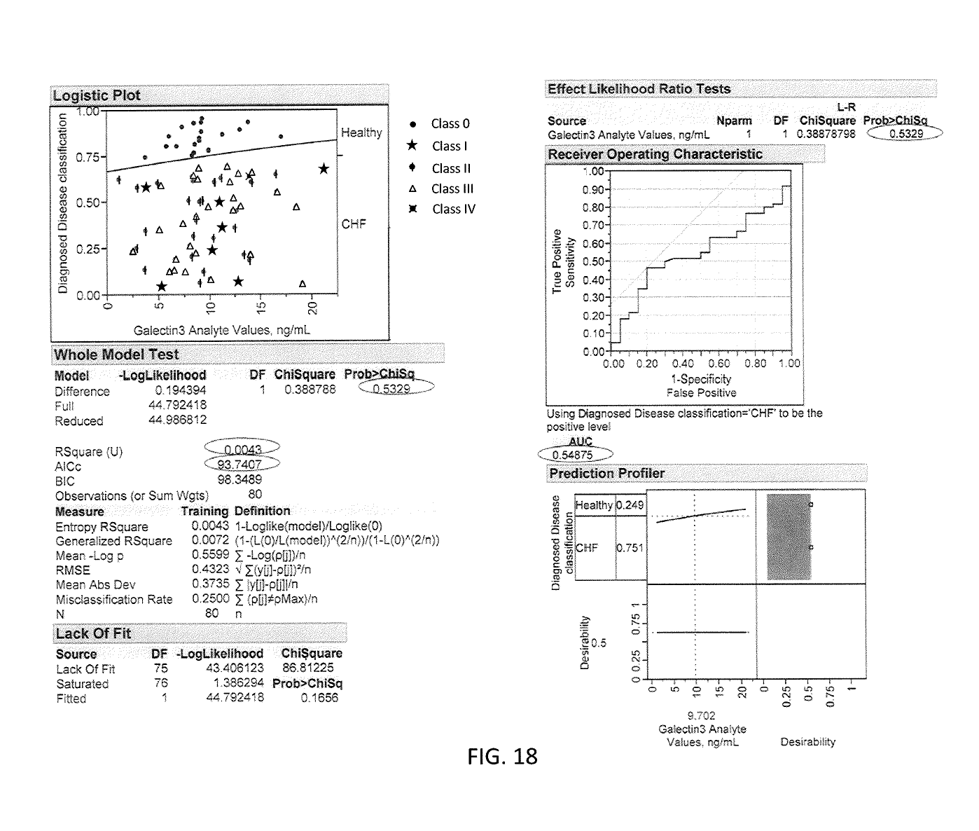

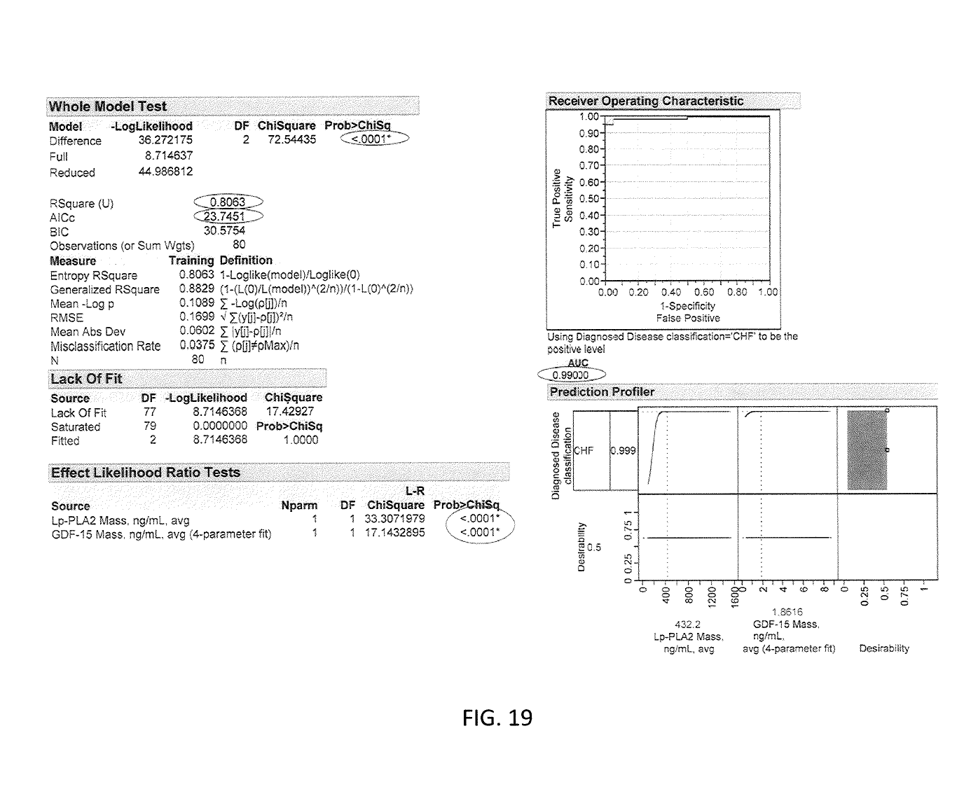

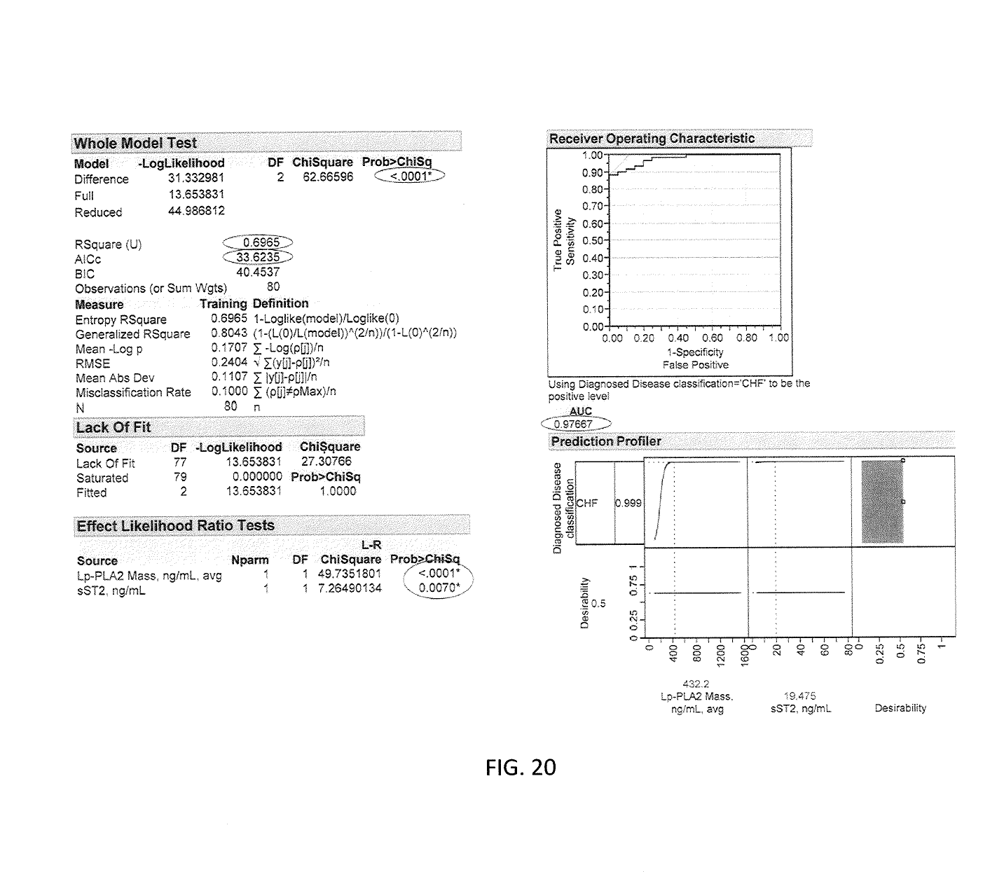

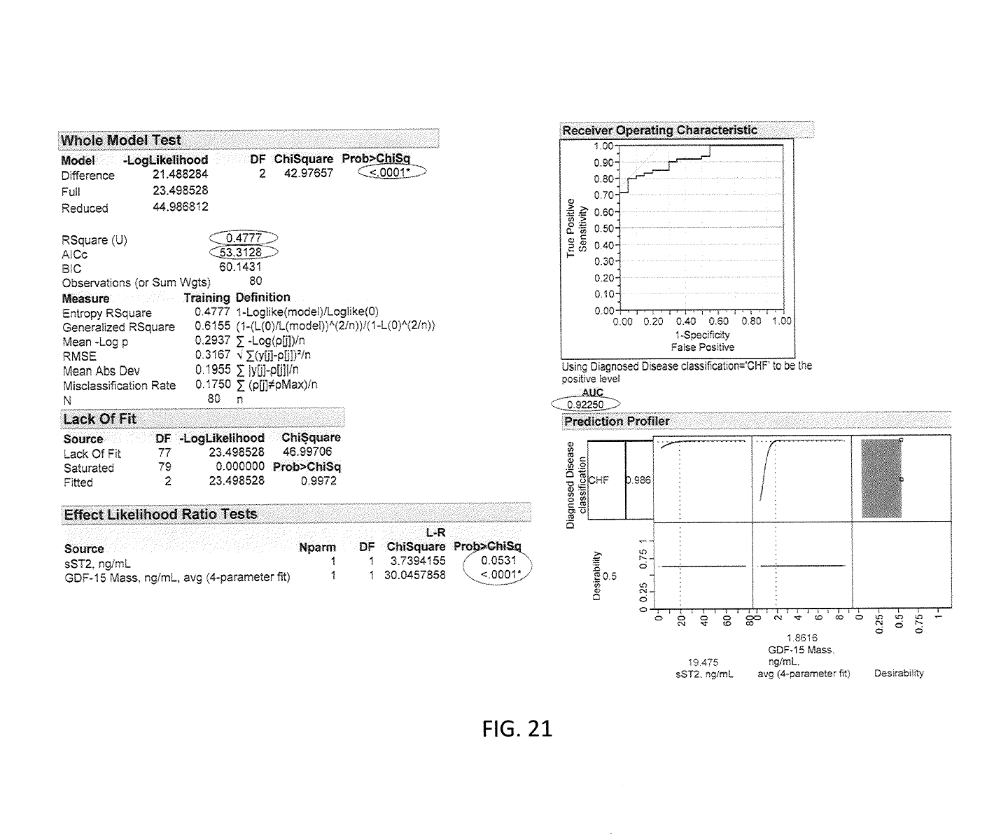

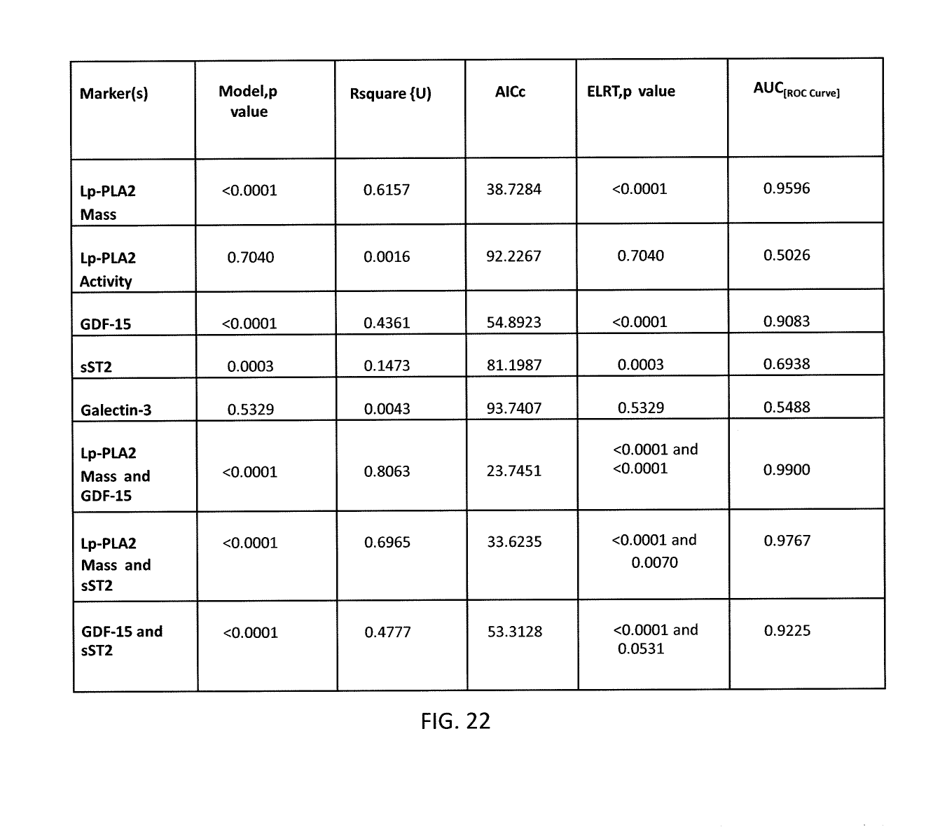

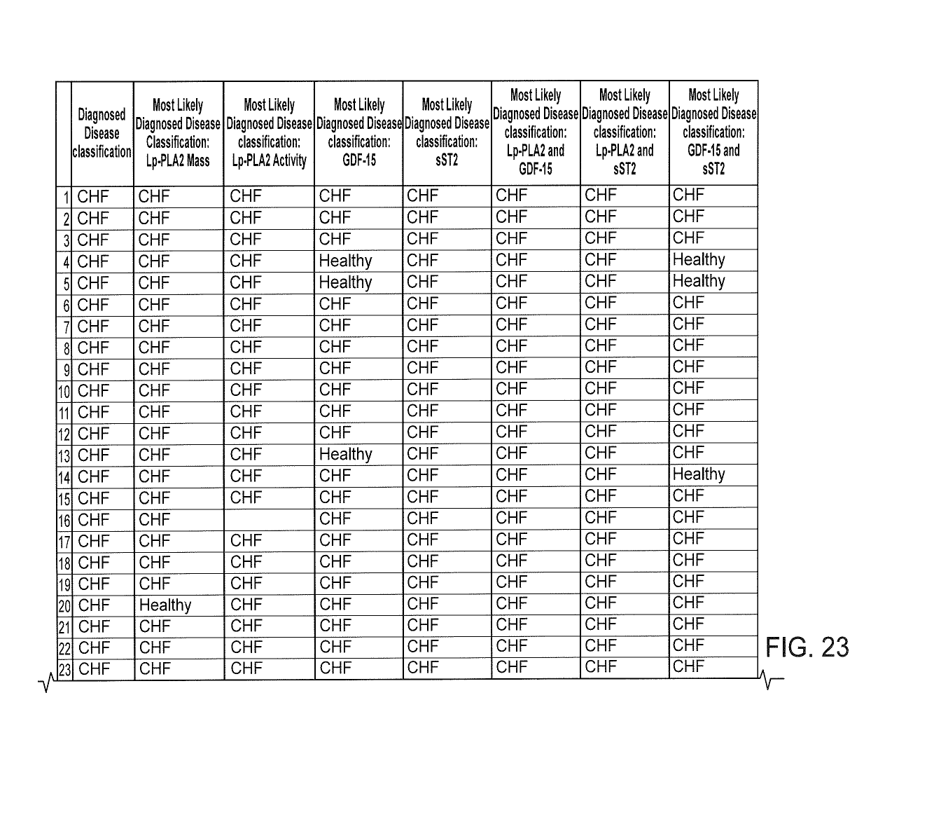

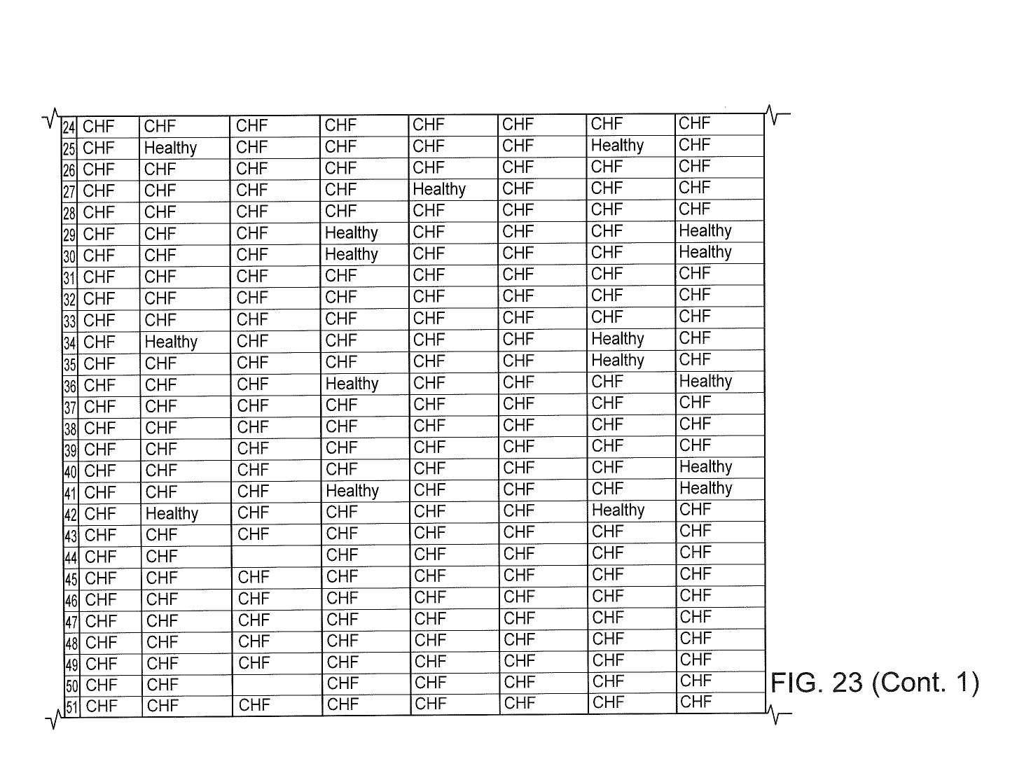

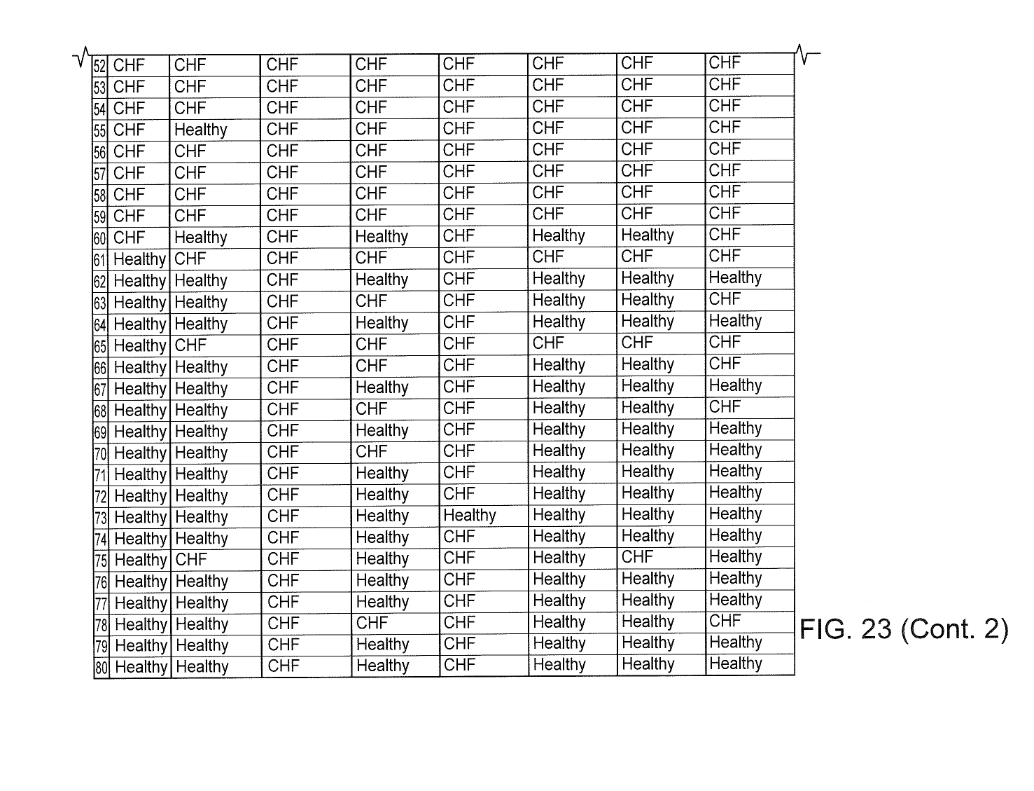

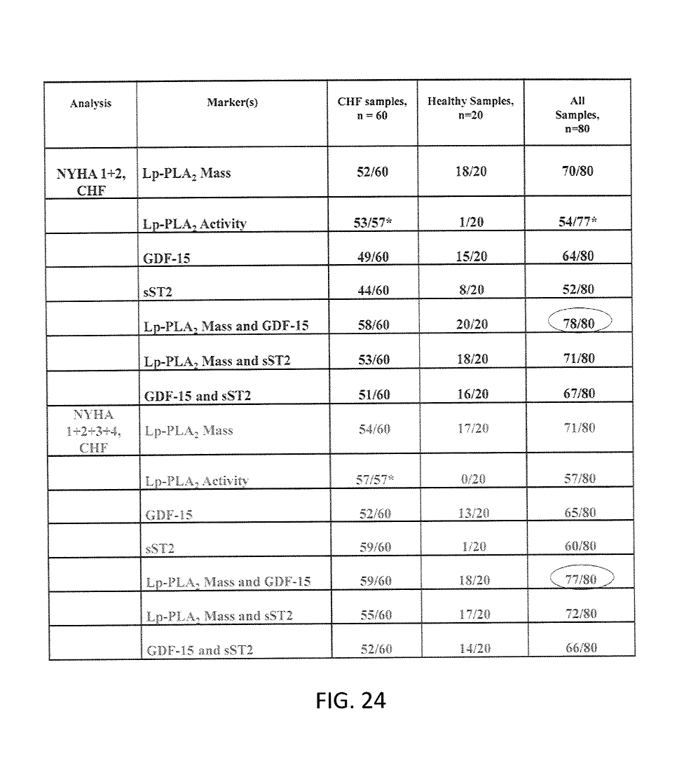

Analyte levels of candidate HF biomarkers were also analyzed on blood plasma from the first Biobank group considering all the samples, including samples from NYHA class 1/2/3/4 and healthy individuals. The nominal logistic regression analysis was performed on eighty samples comprising 60 CHF samples comprising NYHA class 1/2/3/4 with 20 healthy samples as control. See FIGS. 14-21 for individual and combined statistical results and FIG. 22/Table 4 for a summary. Lp-PLA₂^{Mass} assay, but not Lp-PLA₂^{Activity} assay, gave very good modeling results with an R_{squared} of 0.6157 and AUC_{ROC Curve} of 0.9596 for the ELISA version compared to R_{squared} of 0.0016 and AUC_{ROC Curve} 0.5026 for the enzymatic version. The analyte GDF-15 also gave good modeling results with an R_{squared} of 0.4361 and AUC_{ROC Curve} of 0.9083 compared to the other two analytes, sST2 (0.1437 and 0.6938, respectively) and Galectin-3 (0.0043 and 0.5488, respectively). Results from the prediction formula for the second set of samples are shown in FIG. 23. The prediction formula for the combination of Lp-PLA₂^{Mass} correctly predicted 71/80 donor sample's disease state as shown in the summary in FIG. 24. Individually, the Lp-PLA₂^{Mass} assay provided the most accurate prognostic information regarding the earliest stages of heart failure (namely, NYHA Stages 1/2/3/4) among the individual analytes studied here. The Lp-PLA₂^{Mass} assay in combination with GDF-15 gave very good results: an R_{squared} of 0.8063 and an AUC_{ROC Curve} of 0.9900 with excellent Effect Likelihood Ratio Test p-values of 0.0001 (or better) for both analytes. This combination of analytes overall gave superior results to the combination of Lp-PLA₂^{Mass}/sST2 or GDF-15/sST2 which also showed predictive value though at a lower level in this experiment. The prediction formula for the combination of Lp-PLA₂^{Mass} and GDF-15 combination correctly predicted 77/80 donor sample's disease state (FIG. 23/Table 5). These results strongly suggest that the combination of the two analytes, Lp-PLA₂^{Mass} and GDF-15, provide accurate prognostic or diagnostic information using the full NYHA 1/2/3/4 data set of eighty samples. Although any of the values described above for Lp-PLA₂^{Mass} and GDF-15 may be useful for performing a heart failure analysis, in a particular example, Lp-PLA₂^{Mass} (cut-off 430 ng/mL) and GDF-15 (cut-off 1.8 ng/mL), may provide accurate prognostic or diagnostic information using the full NYHA 1/2/3/4 data set of eighty samples. This is also consistent with the combination of PLA₂^{Mass} and GDF-15 analytes being an early prognostic or diagnostic indicator of CHF.

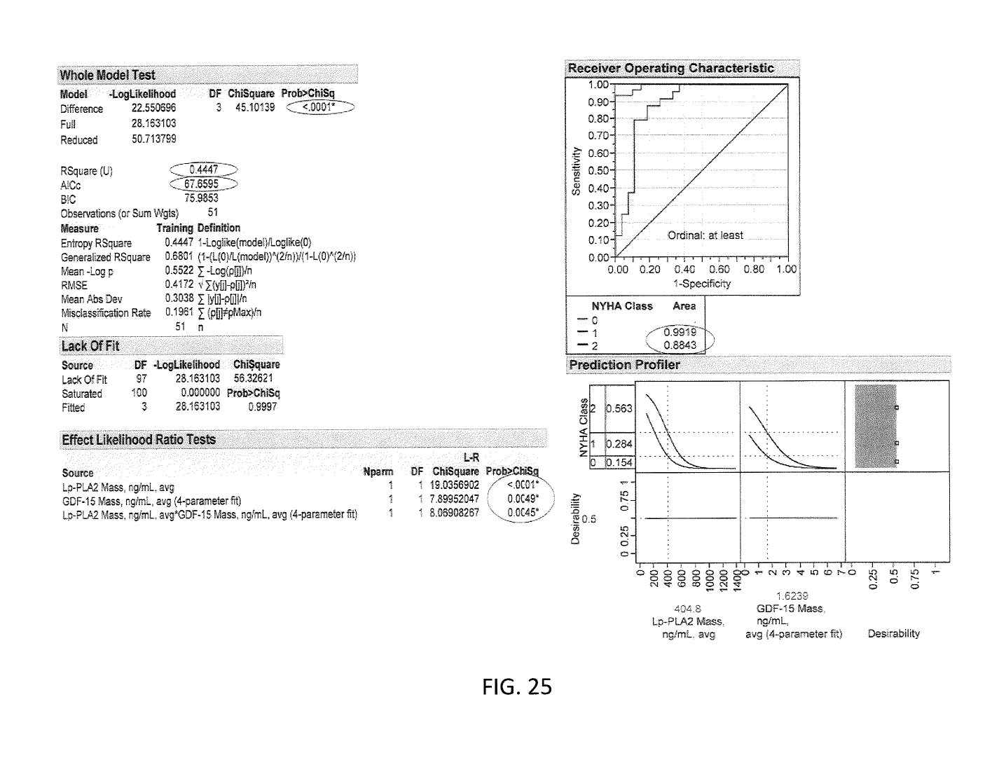

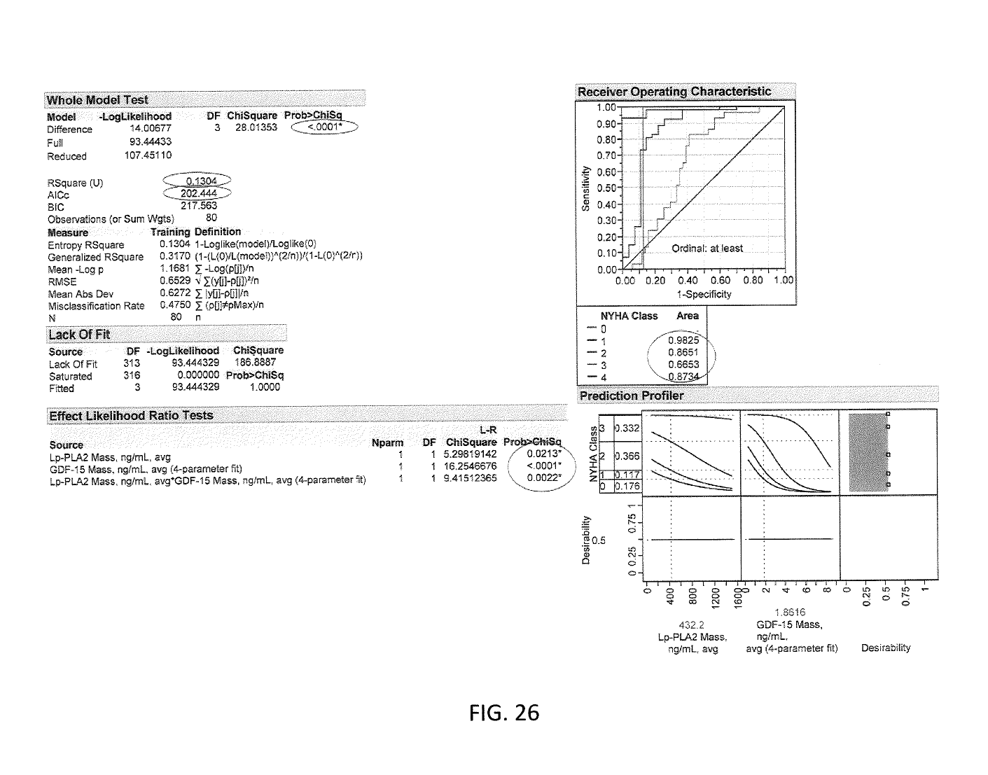

Ordinal logistic regression analyses were performed on the first Biobank sample described above. Fifty-one samples comprising the NYHA class 1/2 and control healthy donor samples (from the above group) were analyzed as were all the samples (NYHA classes 1-4). Consistent with the results of the parsed nominal logistic regression study on NYHA 1/2 samples, the combination of PLA₂^{Mass} and GDF-15 gave more compelling results with the individual NYHA class 1 and class 2 than with the class 3 donor samples. (Compare results FIGS. 25 and 26). Taken together, these results of both the nominal and ordinal logistic regression analyses are consistent with a prominent role for the combination of PLA₂^{Mass} and GDF-15 being an early prognostic or diagnostic indicator of CHF.

The combination of biomarkers Lp-PLA₂^{Mass} and sST2 also shows intriguing promise when they are used together for prognosis of Congestive Heart Failure (see FIGS. 12, 22, and 24; Tables, 2, 4 and 6). Even so, in both analyses (using NYSA 1, 2 or NYHA 1, 2, 3, 4) the combination of Lp-PLA₂^{Mass} and GDF-15 gave superior predictive results (FIG. 24/Table 6).

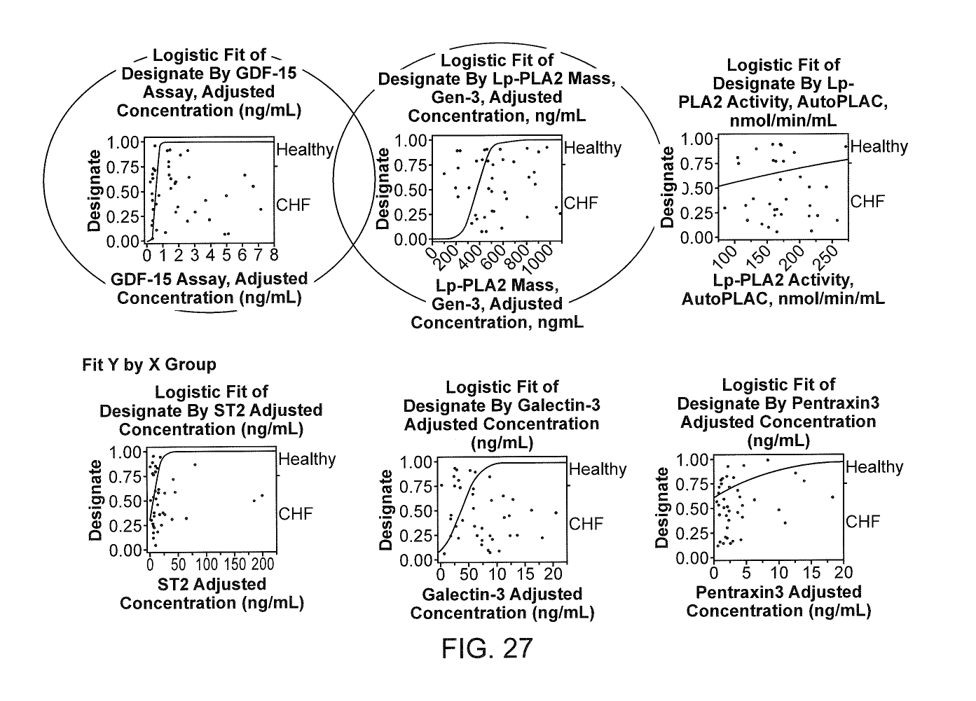

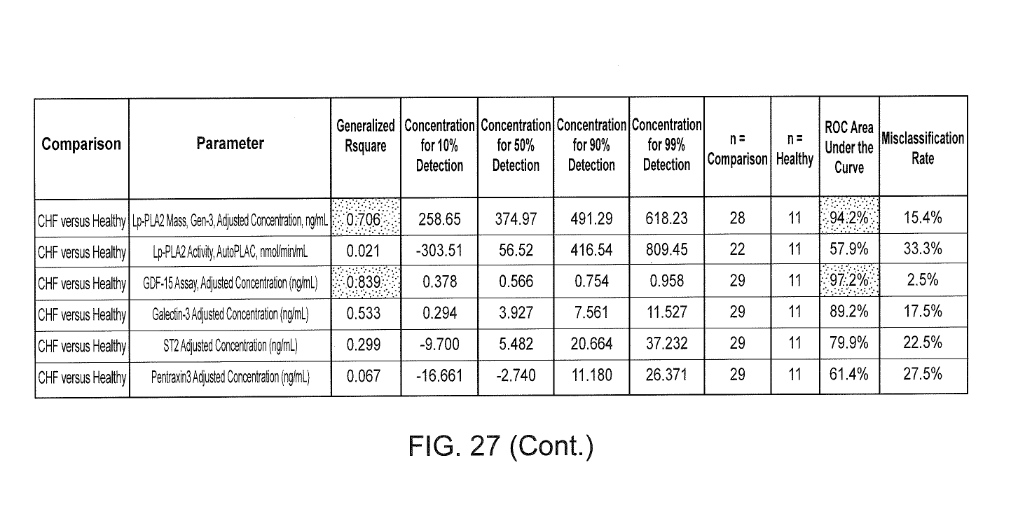

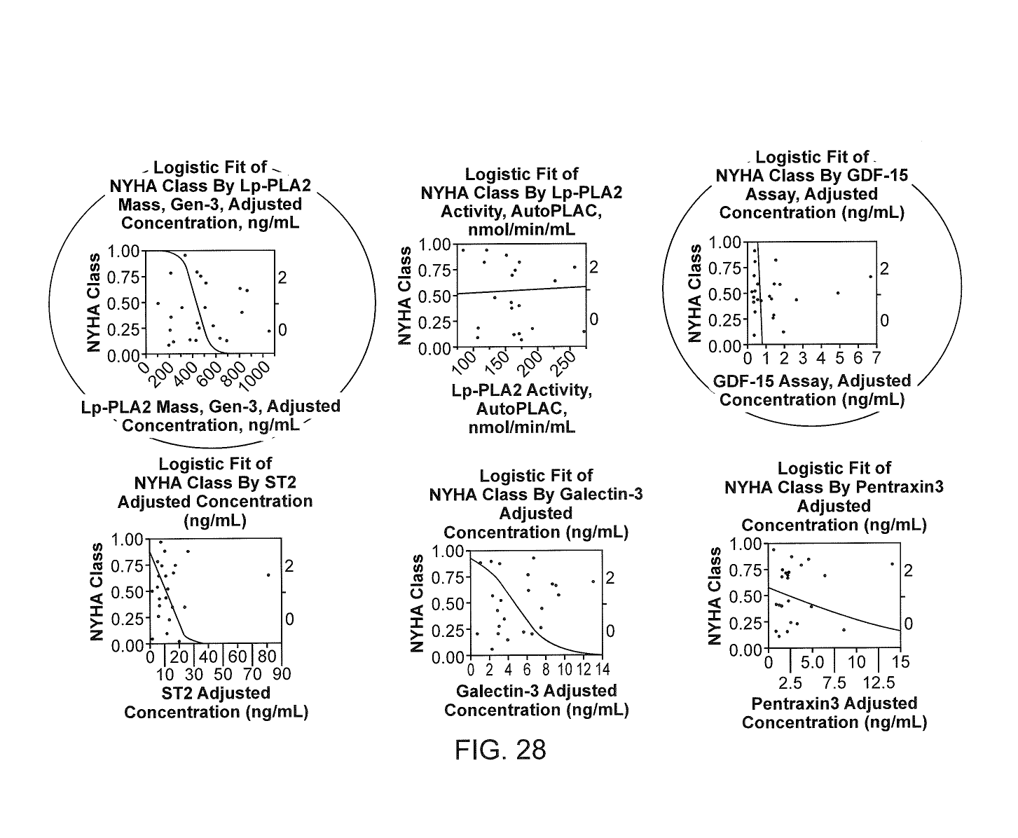

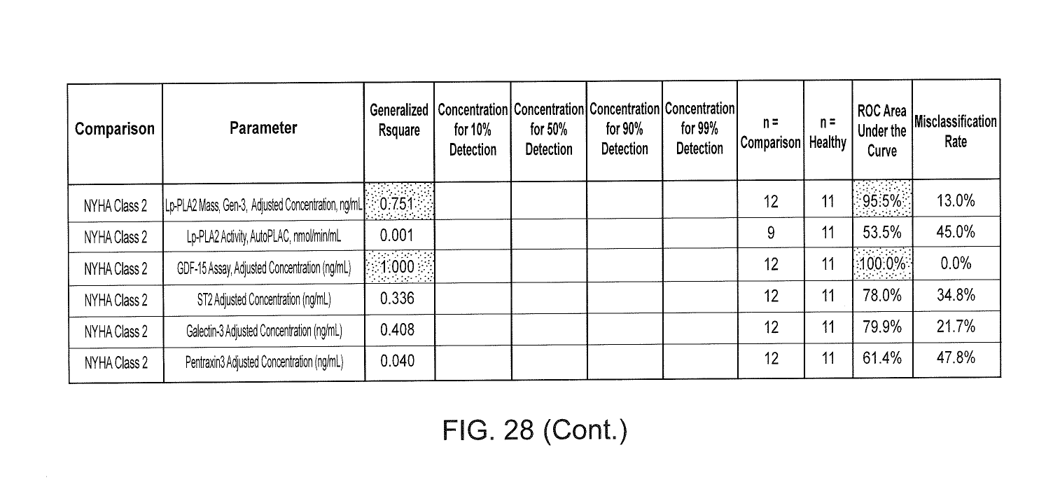

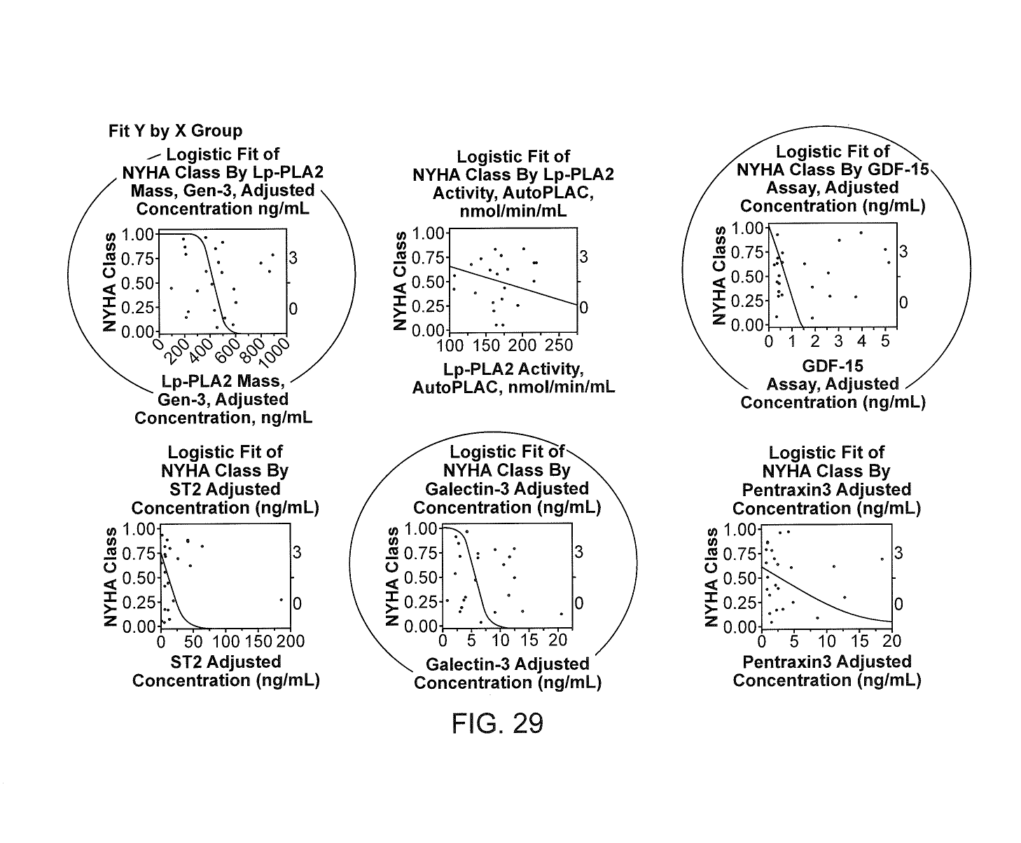

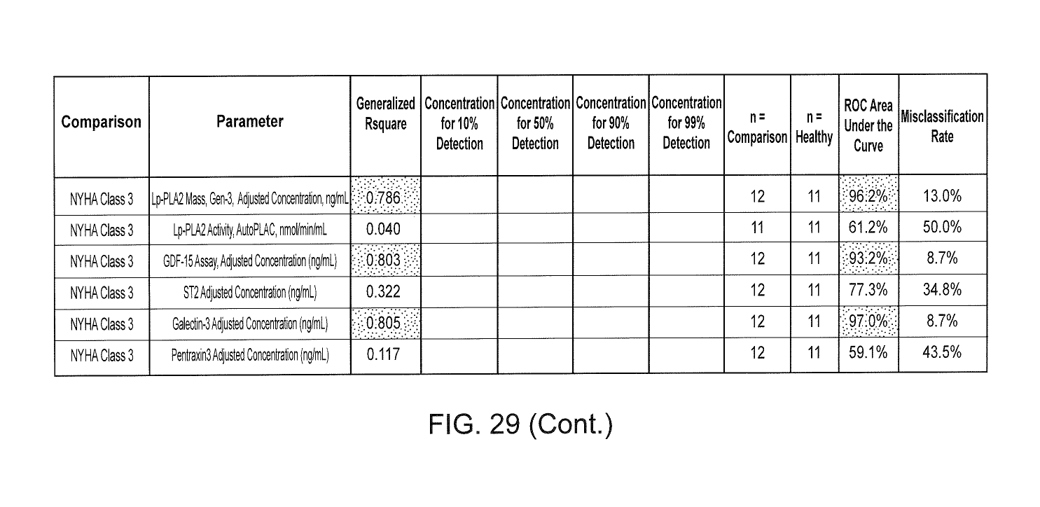

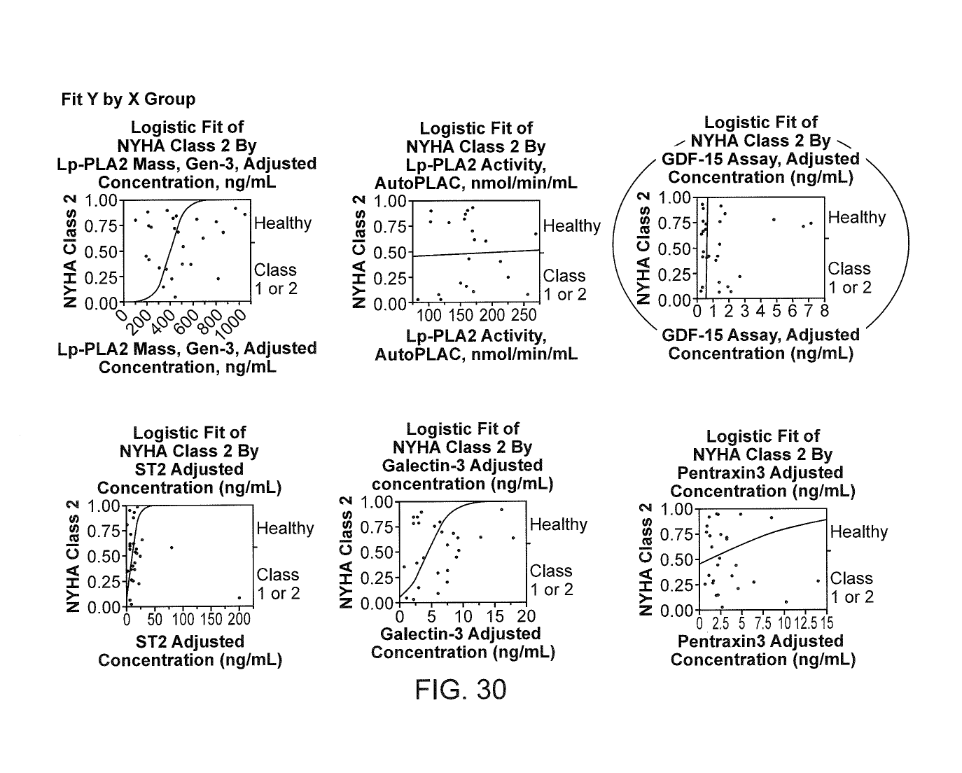

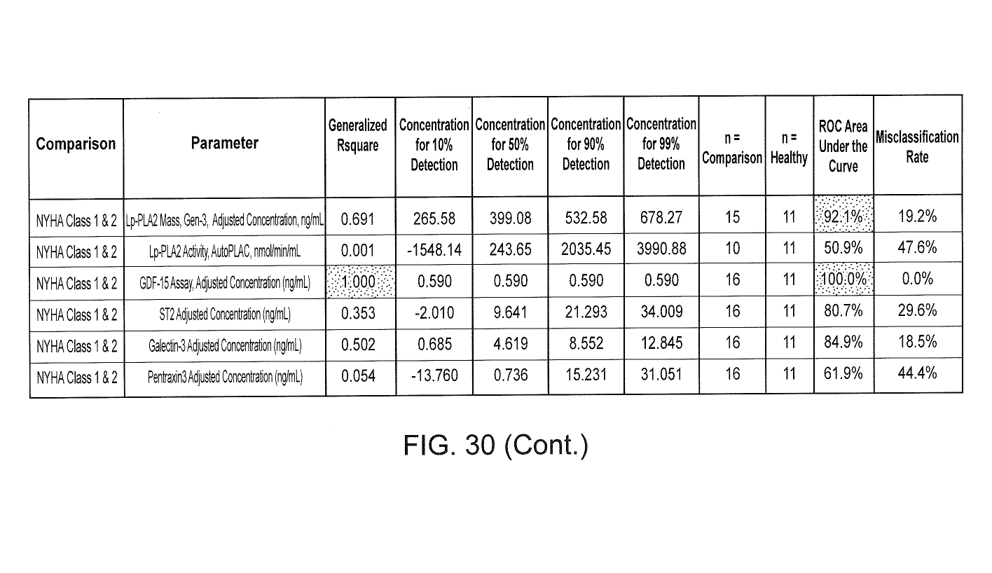

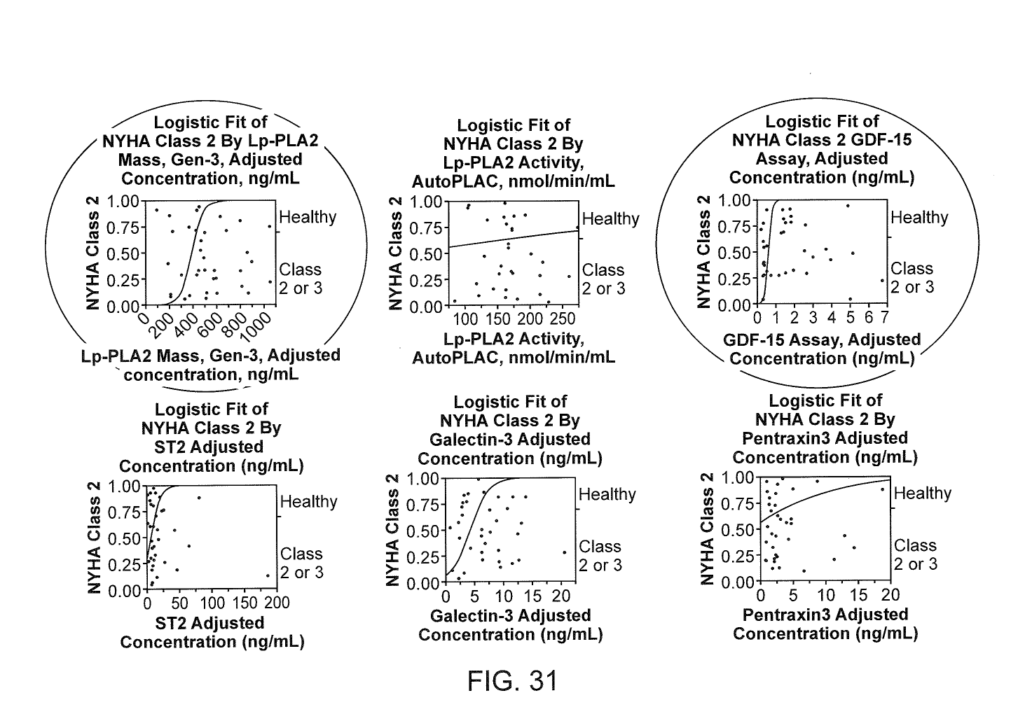

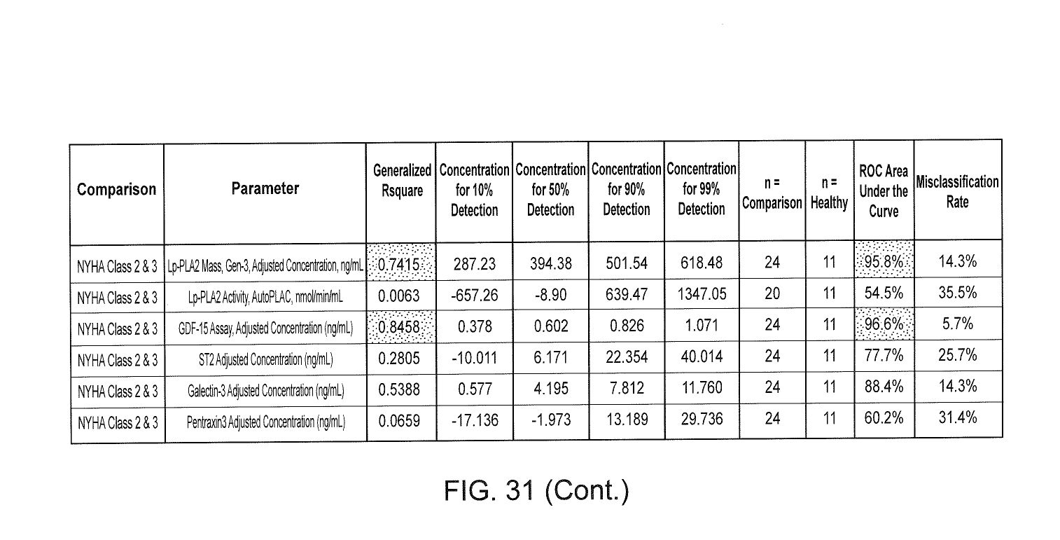

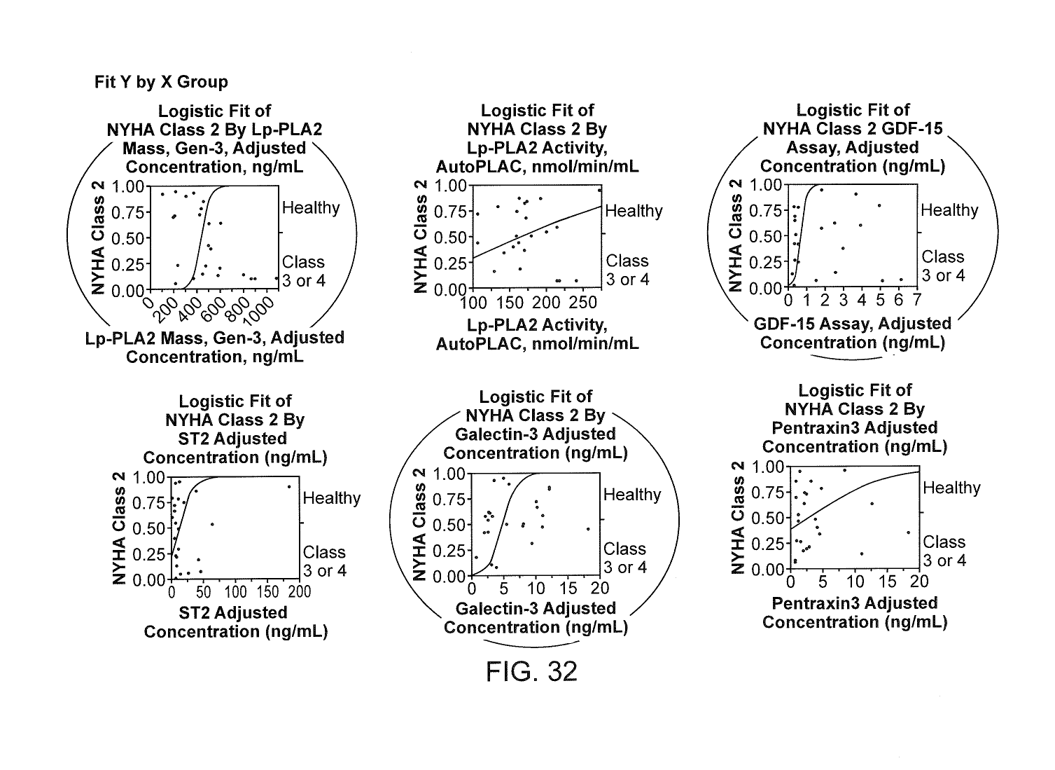

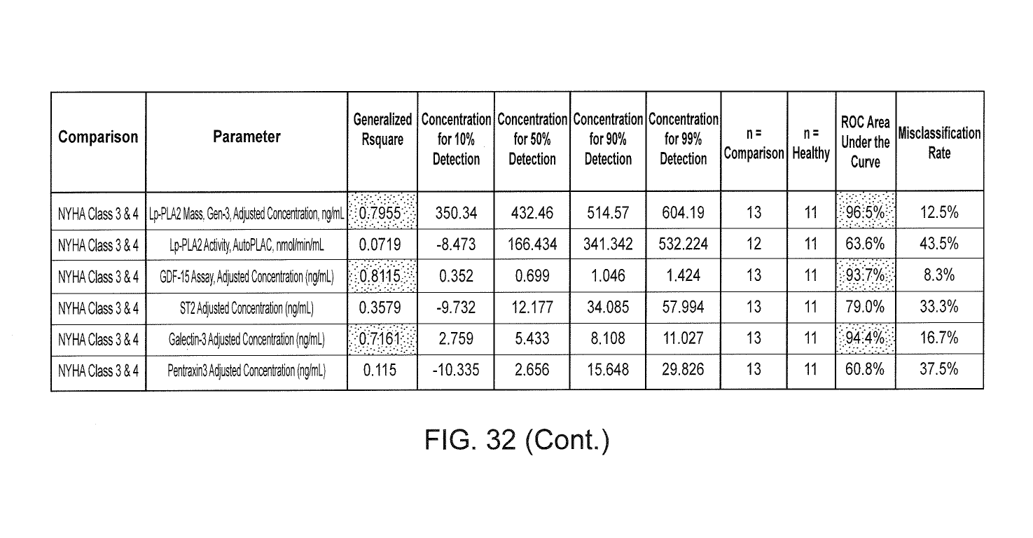

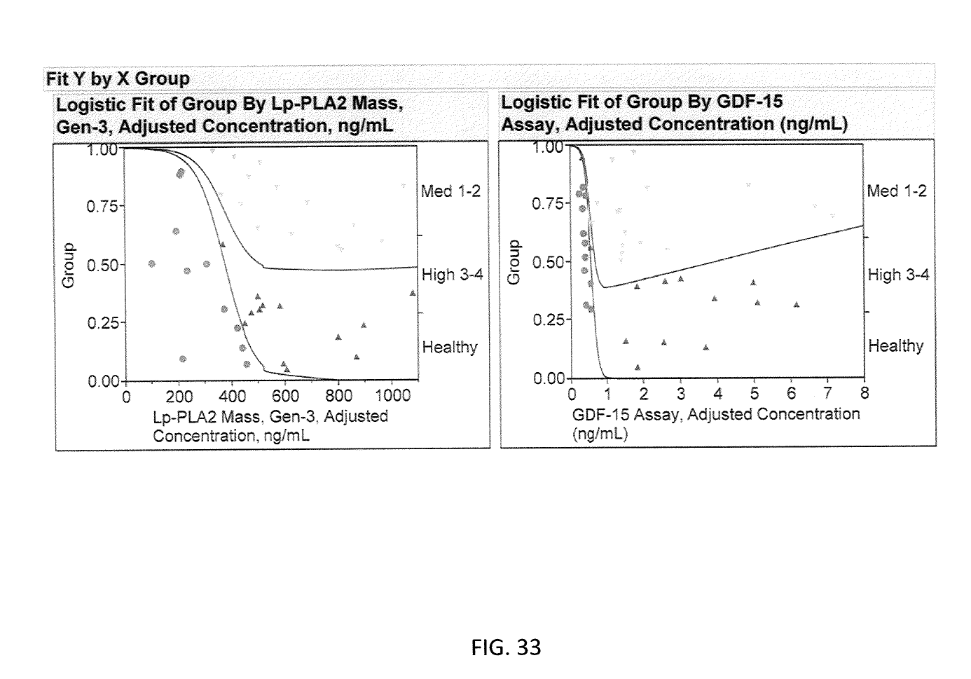

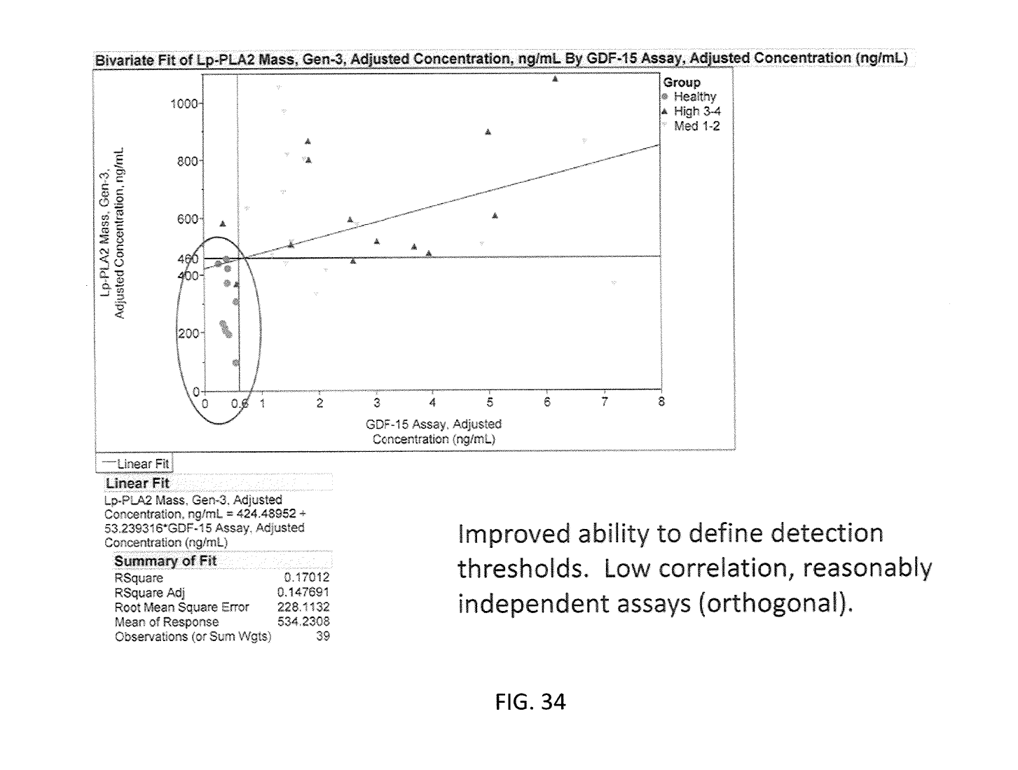

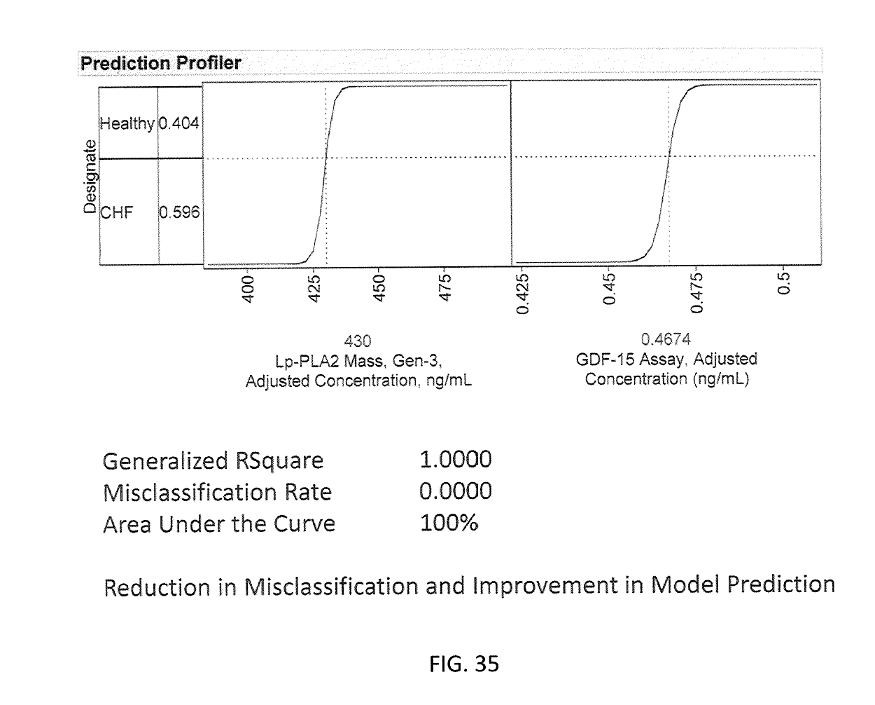

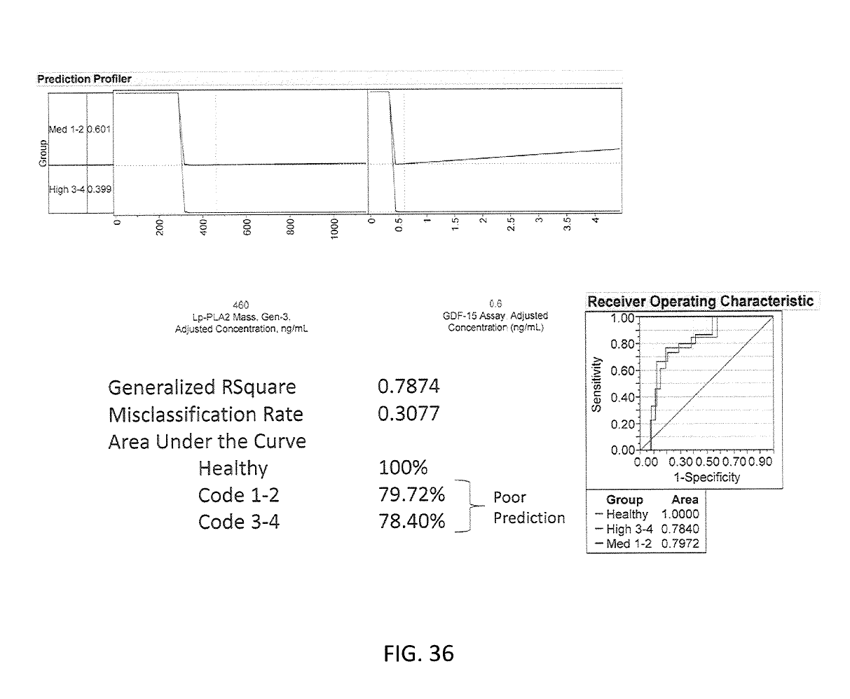

FIGS. 27-36 show statistical analyses of the results of a second, 40-sample Biobank with 29 CHF from classes 1-4 and 11 healthy donors. Individual assays (GDF-15 alone, Lp-PLA₂^{Mass}, and Galectin-3) identified samples with HF vs healthy samples. Statistical analysis using Lp-PLA₂^{Mass} with GDF-15 showed an improved ability to define detection thresholds.

One aspect of the invention may include a method of diagnosing and treating heart failure in an individual including determining a level of a biomarker chosen from the list above in a biological sample from the individual; establishing a diagnosis of heart failure based on the level of the biomarker; and treating the individual with an appropriate therapy for the diagnosed heart failure.

A treatment for heart failure may be any treatment as known in the art such as a medication, use of a medical device, surgery, or another type of treatment. A treatment may include a medication, such as an aldosterone antagonist, an angiotensin-converting enzyme inhibitor, an angiotensin II receptor blocker, a beta blocker, digoxin, a diuretic, an inotrope. A treatment may include a surgery, such as coronary bypass surgery, heart valve repair or replacement, an implantable cardioverter-defibrillator (ICD), cardiac resynchronization therapy, a heart pump, or a heart transplant. Another type of treatment may include, for example, implant of stem cells such as cardiac or other stem cells.

### Treatment Methods

As mentioned above, the techniques described here may be used to treat or prevent heart failure. For example, a method of treating or preventing heart failure (e.g., in a patient previously undiagnosed as having heart failure) may include detecting a level of Lp-PLA2 (e.g., mass) either alone or in combination with one or more other biomarkers (e.g., GDF-15, sST2, etc.) and treating the patient by prescribing a therapy to treat heart failure based on the level of Lp-PLA2, or Lp-PLA2 and one or more other biomarker.

As used herein treating the treating the individual with a therapy for heart failure based on the scored level of risk for heart failure generally means reporting the raw score and/or a ranked or numeric level based on the assay to a health care provider equipped to administer a treatment, e.g., physician, clinician, hospital, clinic, or the like). For example, treating the treating the individual for with a therapy for heart failure based on the scored level of risk for heart failure may include reporting a score for the Lp-PLA2 assay, or a combined score for the Lp-PLA2 assay (e.g., mass assay) and one or more other biomarker levels, such as a level of: a myocyte stretch marker (e.g., GDF-15), sST2, a neurohumoral marker (such as adrenomedullin and endothelin-1), a mid-regional proadrenomedullin (MR proADM), endothelin-1, a marker of myocyte stress (e.g., Atrial natriuretic peptide), a mid-regional pro-atrial natriuretic peptide (MR-proANP), BNP/NTproBNP, necrotic markers (e.g., troponin I/T, CKMB), and/or clinical factors such as age and sex, diabetes, renal clearance or kidney function/dysfunction (creatine, eGFR, NGAL), adrenal function (ACTH/POMC), past ischemic heart disease, heart function (left ventricular ejection fraction/LVEF), and shortness of breath (active signs and symptoms).

Scoring may include scoring and treating may include providing an array (e.g., vector) of raw values for each biomarker (e.g., Lp-PLA2 and one or more of any of the biomarkers described above), a summed value based on the summed values (including weighted summed values) of two or more biomarkers described herein (including Lp-PLA2 and any of the biomarkers described above), and/or ratios of two or more of the biomarkers described herein (e.g., Lp-PLA2 score normalized by two or more biomarkers described herein), products of two or more of the biomarkers described herein (e.g., LP-PLA2 weighted by any of the biomarkers described herein), or the like. When Lp-PLA2 is used as part of the scores, it may refer, in particular, to an estimation of a mass value of Lp-PLA2 taken from a biological sample.

Scoring may also include indicating or correlating the biomarker values determined in to one or more preexisting categories or classifications, including the NYHA classification system described above, and/or the ACC stages: stage A (patients at high risk for developing HF in the future but no functional or structural heart disorder), stage B (a structural heart disorder but no symptoms at any stage); stage C (previous or current symptoms of heart failure in the context of an underlying structural heart problem, but managed with medical treatment); and stage D (advanced disease requiring hospital-based support, a heart transplant or palliative care). Stage A encompasses “pre-heart failure” where intervention with treatment can presumably prevent progression to overt symptoms; stage A does not have a corresponding NYHA class. ACC Stage B would correspond to NYHA Class I, ACC Stage C corresponds to NYHA Class II and III, and ACC Stage D overlaps with NYHA Class IV.

Any of the methods and apparatuses described herein may include the treatment of the individual (e.g., subject or patient) based on the score level of the individual's risk level for heart failure based on the level of Lp-PLA2 (and one or more additional biomarkers). Thus, scoring and/or treatment steps may include categorizing the risk based on low, moderate and high or more or less granular categories, including categories that correlate to existing categories such as NYHA functional classifications. For example, a treatment step may include treating the individual at high risk (e.g., with a classification of NYHA class III/IV) with an appropriate pharmacological agent and/or surgical intervention. A treatment step may include treating an individual having a score indicating a moderate to low risk (e.g., equivalent to an NYHA class I/II) with an appropriate pharmacologic and/or therapy-based treatment.

For example, treatment for an individual having a risk level for heart failure determined by Lp-PLA2 and/or one or more additional biomarkers (as described above) that is “high” and/or equivalent to NYHA Class III/IV may include pharmacological and interventional techniques. Examples of pharmacologic treatments for Class III/IV heart failure (“high” risk) may include: to improve morbidity and mortality: ACE (angiotensin-converting enzyme) inhibitors, ARBs (angiotensin II type I receptor blockers, useful if ACE inhibitor intolerant or plus ACE inhibitors if still symptomatic), selected β-blockers, aldosterone antagonists; to control of symptoms: diuretics (eventually thiazide plus loop diuretic), digitalis (low-dose), temporary inotropics, selected antiarrhythmics; and for palliation: opioids, antidepressants, anxiolytics, oxygen, and continuous inotropics. Treatment of high risk (e.g., type III/IV) may also or alternatively include mechanical and surgical management, including cardiac resynchronisation therapy (CRT) using biventricular pacing, CRT plus implantable cardioverter-defibrillator (ICD), and heart transplantation.

Treatment for an individual having a risk level for heart failure determined by Lp-PLA2 and/or one or more additional biomarkers (as described above) that is “low” or “moderate” and/or equivalent to NYHA Class I/II may include lifestyle changes and medications. Examples may include: 2-g Sodium diet, daily monitoring, managing weight, fluid restriction, monitoring blood pressure, smoking cessation, light aerobic exercise, and medications (e.g., blood thinners, angiotensin converting enzyme inhibitor (ACE inhibitor) or angiotensin II receptor blocker (ARB), beta blocker). Surgical interventions may be indicated, including coronary artery repair and valve repair or replacement, as appropriate.

Any appropriate therapy may be used, but particularly a pharmaceutical agent (e.g., drug, compound, composition). Examples of such pharmaceutical agents includes: angiotensin-converting enzyme (ACE) inhibitors, angiotensin-receptor blockers (ARBs), Beta blockers, diuretics, aldosterone blockers, digitalis, hydralazine and nitrates, statins, aspirin and warfarin.

Angiotensin-converting enzyme (ACE) inhibitors are often used for treating patients with heart failure. ACE inhibitors open blood vessels and decrease the workload of the heart. They are used to treat high blood pressure but can also help improve heart and lung muscle function. ACE inhibitors are particularly important for patients with diabetes, because they also help slow progression of kidney disease.

Angiotensin-Receptor Blockers (ARBs), also known as angiotensin II receptor antagonists, are similar to ACE inhibitors in their ability to open blood vessels and lower blood pressure. They may have fewer or less-severe side effects than ACE inhibitors, especially coughing, and are sometimes prescribed as an alternative to ACE inhibitors. Some patients with heart failure take an ACE inhibitor along with an ARB.

Another class of drugs that may be used to treat heart failure includes Angiotensin Receptor Neprilysin Inhibitors (ARMs) (e.g., a combination of sacubitril and valsartan). These drugs may be used in place of ACE/ARBs, while continuing to use other drugs, such as beta blockers and MRAs.

Beta blockers are almost always used in combination with other drugs, such as ACE inhibitors and diuretics. They help slow heart rate and lower blood pressure. When used properly, beta blockers can reduce the risk of death or rehospitalization. Beta blockers can lower HDL (“good”) cholesterol, so have not previously been used with patients having a high level of Lp-PLA2.

Diuretics cause the kidneys to rid the body of excess salt and water. Fluid retention is a major symptom of heart failure. Aggressive use of diuretics can help eliminate excess body fluids, while reducing hospitalizations and improving exercise capacity. These drugs are also important to help prevent heart failure in patients with high blood pressure. In addition, certain diuretics, notably spironolactone (Aldactone), block aldosterone, a hormone involved in heart failure. This drug class is beneficial for patients with more severe heart failure (Stages C and D). Patients taking diuretics usually take a daily dose. Diuretics, or any of the treatments described herein, may be modified based on the level of Lp-PLA2 or Lp-PLA2 in combination with one or more other biomarkers. For example, the amount and timing of the diuretic (or other heart failure agent) may be adjusted on this basis.

Aldosterone is a hormone that is critical in controlling the body's balance of salt and water. Excessive levels may play important roles in hypertension and heart failure. Drugs that block aldosterone are prescribed for some patients with symptomatic heart failure. They have been found to reduce mortality or death rates for patients with heart failure and coronary artery disease, especially after a heart attack. These blockers pose some risk for high potassium levels.

Digitalis is derived from the foxglove plant. It has been used to treat heart disease since the 1700s. Digoxin (Lanoxin) is the most commonly prescribed digitalis preparation. Digoxin decreases heart size and reduces certain heart rhythm disturbances (arrhythmias). Unfortunately, digitalis does not reduce mortality rates, although it does reduce hospitalizations and worsening of heart failure. Controversy has been ongoing for more than 100 years over whether the benefits of digitalis outweigh its risks and adverse effects. Digitalis may be useful for select patients with left-ventricular systolic dysfunction who do not respond to other drugs (diuretics, ACE inhibitors). It may also be used for patients who have atrial fibrillation.

Hydralazine and nitrates are two older drugs that help relax arteries and veins, thereby reducing the heart's workload and allowing more blood to reach the tissues. They are used primarily for patients who are unable to tolerate ACE inhibitors and angiotensin receptor blockers. In 2005, the FDA approved BiDil, a drug that combines isosorbide dinitrate and hydralazine. BiDil is approved to specifically treat heart failure in African-American patients.

Statins are important drugs used to lower cholesterol and to prevent heart disease leading to heart failure. These drugs include lovastatin (Mevacor), pravastatin (Pravachol), simvastatin (Zocor), fluvastatin (Lescol), atorvastatin (Lipitor), and rosuvastatin (Crestor). In 2007, the Food and Drug Administration (FDA) approved atorvastatin to reduce the risks for hospitalization for heart failure in patients with heart disease.

Aspirin is a type of non-steroid anti-inflammatory (NSAID). Aspirin is recommended for preventing death in patients with heart disease, and can safely be used with ACE inhibitors, particularly when it is taken in lower dosages (75-81 mg).

In particular, the techniques described herein may be used to treat a subject by providing aspirin (e.g., acetylsalicylic acid) when the subject's level of Lp-PLA2 exceeds a threshold (e.g., >about 400 ng/ml) alone or in combination with one or more other biomarkers. Curiously, previous work has taught away from the use of aspirin when the level of Lp-PLA2 is above normal in patients. See, e.g., Hatoum et al. “Dietary, lifestyle, and clinical predictors of lipoprotein-associated phospholipase A2 activity in individuals without coronary artery disease” in Am J Clin Nutr 2010; 91:786-93. (“Aspirin use was also positively associated with Lp-PLA2 activity”).

Warfarin (Coumadin) is recommended only for patients with heart failure who also have: atrial fibrillation, a history of blood clots to the lungs, stroke, or transient ischemic attack, a blood clot in one of their heart chambers. Other drugs that may be used may include Nesiritide (Natrecor), Erythropoietin, Tolvaptan, Levosimendan, etc.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein.

Any of the biomarkers described herein may be included in an apparatus, such as an assay, panel, or the like. In particular, any of the biomarkers described herein may be used as a part of a panel configured specifically to determine early risk (e.g., class I/II) for heart failure, including in patients not yet diagnosed with appreciable risk. Assays may include Lp-PLA2 as well as one or more additional biomarkers such as GDF-15, sST1, etc. (including any of the biomarkers described herein). In some variations the assays are configured to determine a level of each biomarker in parallel.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A method of treating heart failure in an individual, the method comprising:
determining a level of Lp-PLA₂ in a biological sample from the individual;
scoring a level individual's risk level for heart failure based on the level of Lp-PLA₂ determined; and
treating the individual with a therapy for heart failure based on the scored level of risk for heart failure.

2. The method of claim 1, wherein the individual has not previously been diagnosed with heart failure.

3. The method of claim 1, wherein determining a level of Lp-PLA₂ in a biological sample from the individual comprises determining a mass level of Lp-PLA₂.

4. The method of claim 1, further comprising determining the level of a myocyte stretch marker and wherein scoring the level individual's risk level for heart failure is based on the level of Lp-PLA2 determined and the level of the myocyte stretch marker in the biological sample.

5. The method of claim 1, further comprising determining the level of GDF-15 and wherein scoring the level individual's risk level for heart failure is based on the level of Lp-PLA₂ determined and the level of the GDF-15 in the biological sample.

6. The method of claim 1, further comprising determining the level of sST2 and wherein scoring the level individual's risk level for heart failure is based on the level of Lp-PLA₂ determined and the level of the sST2 in the biological sample.

7. The method of claim 1, further comprising determining the level of a neurohumoral marker and wherein scoring the level individual's risk level for heart failure is based on the level of Lp-PLA₂ determined and the level of the neurohumoral marker in the biological sample.

8. The method of claim 1, further comprising determining the level of a mid-regional proadrenomedullin (MR proADM) and wherein scoring the level individual's risk level for heart failure is based on the level of Lp-PLA₂ determined and the level of MR proADM in the biological sample.

9. The method of claim 1, further comprising determining the level of a endothelin-1 and wherein scoring the level individual's risk level for heart failure is based on the level of Lp-PLA₂ determined and the level of the endothelin-1 in the biological sample.

10. The method of claim 1, further comprising determining the level of a myocyte stress marker and wherein scoring the level individual's risk level for heart failure is based on the level of Lp-PLA₂ determined and the level of the myocyte stress in the biological sample.

11. The method of claim 1, further comprising determining the level of a mid-regional pro-atrial natriuretic peptide (MR-proANP) and wherein scoring the level individual's risk level for heart failure is based on the level of Lp-PLA₂ determined and the level of the MR-proANP in the biological sample.

12. The method of claim 1, further comprising determining the level of a BNP/NTproBNP and wherein scoring the level individual's risk level for heart failure is based on the level of Lp-PLA₂ determined and the level of the BNP/NTproBNP in the biological sample.

13. The method of claim 1, further comprising determining the level of a necrotic marker and wherein scoring the level individual's risk level for heart failure is based on the level of Lp-PLA₂ determined and the level of the necrotic marker in the biological sample.

14. The method of claim 1, further comprising determining the level of a troponin I, troponin T, or troponin I and troponin T and wherein scoring the level individual's risk level for heart failure is based on the level of Lp-PLA₂ determined and the level of the troponin I, troponin T, or troponin I and troponin T in the biological sample.

15. The method of claim 1, further comprising determining the level of a creatine kinase MB (CK-MB) and wherein scoring the level individual's risk level for heart failure is based on the level of Lp-PLA₂ determined and the level of the CK-MB in the biological sample.

16. The method of claim 1, wherein scoring comprising scoring an individual with risk level for heart failure when the individual has a mass value for Lp-PLA₂ between about 400 ng/ml and about 600 ng/ml at a moderate risk for heart failure.

17. The method of claim 1, wherein scoring comprising scoring an individual with risk level for heart failure when the individual has a mass value for Lp-PLA₂ above about 600 ng/ml at a high risk for heart failure.

18. The method of claim 4, wherein scoring comprising scoring an individual as at risk for heart failure when the individual has a level of Lp-PLA₂ greater than about 400 ng/ml and a level of GDF-15 above about 1.6 ng/mL.

19. The method of claim 1, wherein treating the individual based on the scored level of risk for heart failure comprises prescribing one or more pharmacological agents selected from the group consisting of: angiotensin-converting enzyme (ACE) inhibitors, angiotensin-receptor blockers (ARBs), Beta blockers, diuretics, aldosterone blockers, digitalis, hydralazine and nitrates, statins, aspirin and warfarin.

20. A method of treating heart failure in an individual, the method comprising:
determining a level of LP-PLA₂ and a second biomarker in a biological sample from the individual;
scoring the level individual's risk level for heart failure based on the level of Lp-PLA₂ and the level of the second biomarker determined; and
treating the individual with a therapy for heart failure based on the scored level of risk for heart failure.

21. The method of claim 20, wherein determining a level of Lp-PLA₂ in a biological sample from the individual comprises determining a mass level of Lp-PLA₂.

22. The method of claim 20, wherein determining the level of the second biomarker comprises determining a level of GDF-15 in the biological sample.

23. The method of claim 20, wherein determining the level of the second biomarker comprises determining a level of mid-regional proadrenomedullin (MR proADM) in the biological sample.

24. The method of claim 20, wherein determining the level of the second biomarker comprises determining a level of endothelin-1 in the biological sample.

25. The method of claim 20, wherein determining the level of the second biomarker comprises determining a level of a myocyte stress marker in the biological sample.

26. The method of claim 20, wherein determining the level of the second biomarker comprises determining a level of mid-regional pro-atrial natriuretic peptide (MR-proANP) in the biological sample.

27. The method of claim 20, wherein scoring comprising scoring an individual with risk level for heart failure when the individual has a mass value for Lp-PLA₂ between about 400 ng/ml and about 600 ng/ml at a moderate risk for heart failure.

28. The method of claim 20, wherein scoring comprising scoring an individual with risk level for heart failure when the individual has a mass value for Lp-PLA₂ above about 600 ng/ml at a high risk for heart failure.

29. The method of claim 20, wherein scoring comprising scoring an individual as at risk for heart failure when the individual has a level of Lp-PLA₂ greater than about 400 ng/ml and a level of GDF-15 above about 1.6 ng/mL.

30. A method of treating heart failure in an individual that has not previously been diagnosed with heart failure, the method comprising:
determining a level of LP-PLA₂^{(Mass)} and a level of GDF-15 in a biological sample from the individual;
scoring the level individual's risk level for heart failure based on the level of Lp-PLA₂ and the level of the second biomarker determined; and
treating the individual with a therapy for heart failure based on the scored level of risk for heart failure.
